(19) United States
(12) Patent Application Publication
(10) Pub. No.: US 2022/0404356 A1
(43) Pub. Date: Dec. 22, 2022

(54) IN VITRO METHOD FOR DETERMINING THE LIKELIHOOD OF OCCURRENCE OF AN ACUTE MICROVASCULAR REJECTION (AMVR) AGAINST A RENAL ALLOGRAFT IN AN INDIVIDUAL

(71) Applicants: INSTITUT NATIONAL DE LA SANTE ET DE LA RECHERCHE MEDICALE (INSERM), Paris (FR); CENTRE NATIONAL DE LA RECHERCHE SCIENTIFIQUE (CNRS), Paris (FR); UNIVERSITE DE PARIS, Paris (FR); CENTRE HOSPITALIER UNIVERSITAIRE DE NANTES, Nantes (FR); ASSISTANCE PUBLIQUE - HOPITAUX DE PARIS, Paris (FR); UNIVERSITE DE NANTES, Nantes (FR)

(72) Inventors: Dany ANGLICHEAU, Paris (FR); Beatrice CHARREAU, Nantes Cedex (FR)

(21) Appl. No.: 17/421,987

(22) PCT Filed: Jan. 10, 2020
(86) PCT No.: PCT/EP2020/050602

§ 371 Date: Jul. 9, 2021

(30) Foreign Application Priority Data
Jan. 11, 2019 (EP) 19305037.4

(51) Int. Cl. G01N 33/564; G01N 33/50; G01N 33/68

(52) U.S. Cl. CPC G01N 33/564; G01N 33/5064; G01N 33/6854; G01N 33/6893; G01N 2800/245

(57) ABSTRACT

The present invention relates to the field of organ transplant and the issues associated with transplant rejection. Anti-body-mediated rejection (AMR) is associated with a poor transplant outcome. Pathogenic alloantibodies are usually directed against human leucocyte antigens (HLAs). However, evidence of AMR in the absence of anti-HLA antibodies suggests the presence of non-anti-HLA antibodies, identified as anti-endothelial cell antibodies (AECAs). The inventors have demonstrated that kidney recipients who experienced acute rejection with microvascular inflammation within the first 3 months after transplantation in the absence of anti-HLA donor-specific antibodies, carried, before transplantation, unknown AECAs in their sera that specifically targeted the glomerular microvascular endothelium. Thus, the present invention relates to in vitro methods and kits for determining the likelihood of occurrence of an acute microvascular rejection (AMVR) against a renal allograft in an individual.

### FIELD OF THE INVENTION

The present invention relates to the field of organ transplant and the issues associated with transplant rejection. In particular, the present invention relates to in vitro methods for determining the likelihood of occurrence of an acute microvascular rejection (AMVR) against a renal allograft in an individual.

The present invention further relates to kits for determining the likelihood of occurrence of an acute microvascular rejection (AMVR) against a renal allograft in an individual.

### BACKGROUND OF THE INVENTION

Transplant rejection occurs when transplanted tissue is rejected by the recipient's immune system. It is an adaptive immune response via cellular immunity (mediated by cytotoxic T cells inducing apoptosis of target cells) as well as humoral immunity (mediated by activated B cells secreting antibody molecules). The present invention focuses essentially on the latter.

Different types of transplanted tissues tend to favour different balances of rejection mechanisms.

It has however been found that rejection can be lessened by determining the molecular similitude between donor and recipient and by use of immunosuppressant drugs after transplant.

Despite the development of potent immunosuppressive regimens, antibody-mediated rejection (AMR), which is associated with a poor transplant outcome, remains a significant hurdle to long-term organ acceptance. AMRs are typically associated with microvascular inflammation of the graft and the presence of antibodies targeting the anti-human leukocyte antigen (HLA) molecules of the transplanted organ (Donor Specific Antibody, DSA).

Even though histological findings suggestive of microvascular inflammation usually demonstrate an anti-human leukocyte antigen (HLA)-mediated injury, a subset of patients develop such lesions in the absence of detectable anti-HLA donor-specific antibodies (DSAs).

Antibody-mediated rejection (AMR) is recognized as a diagnostic entity and is considered a major cause of late kidney allograft failure. Significant progress has been made in the development of sensitive assays for the detection of donor-specific antibodies (DSA) against human leukocyte antigens (HLA). Initially, the three main diagnostic features of AMR were (1) morphological evidence of tissue injury, (2) presence of DSA and (3) complement split product 4d (C4d) staining in peritubular capillaries as a footprint for complement-mediated injury (Senev at al., 2018, Am. J Transplant, 00: 1-18).

However, in clinical practice, not all cases fulfil the above-cited three criteria. Patients with such an incomplete phenotype are classified as “suspicious for AMR”. These poorly studied cases may essentially be classified into two categories, namely (1) patients with DSA and some histological lesions of AMR, but not meeting the full histologic criteria for AMRE and (2) patients meeting the histological criteria for AMR but without detectable DSA.

Thus, the occurrence of non-anti-HLA-associated AMRs remains associated with unresolved diagnostic and therapeutic issues.

The potential involvement of non-HLA antibodies (Abs) in renal allograft rejection is mentioned by the current Banff classification, which mentions the presence of “serologic evidence of DSA against HLA or other antigens”. This classification of allograft pathology has provided framework for the reporting of renal allograft biopsies and has answered the need for an international consensus on renal transplant biopsy reporting, providing guidance for clinical diagnosis and enabling meaningful comparison between research studies and clinical trials investigating the diagnosis, treatment and outcome in kidney transplantation.

The involvement of non-HLA antibodies has also been considered in the prior art.

However, in the absence of clearly defined antigens, the assumption that acute rejections with significant microvascular inflammation (called AMVRs thereafter, for acute microvascular rejections) are true AMRs remains hypothetical.

What's more, although this issue is of utmost importance for treatment decisions, it may be difficult to demonstrate that the observed graft injury is induced by Abs.

These particular types of immune injuries are presumed to be due to Abs reactive to non-HLA antigens expressed on endothelial cells (ECs). These Abs might be alloantibodies directed against non-HLA polymorphic antigens that differ between the recipient and donor or autoantibodies that recognize self-antigens consequent to a breakdown of self-tolerance (Reindl-Schwaighofer et al., Mechanisms underlying human genetic diversity: consequence for antigraft antibody responses. Trampl Int, 31: 239-250, 2018).

Thus, there remains a need for improving diagnosis of AMR in transplanted patients, including in recipients of a renal allograft.

In particular, there remains a need for the provision of a method allowing the prediction or the diagnosis of an antibody-mediated acute allograft rejection to which contribute anti-endothelial cells antibodies (AECAs) directed to non-HLA antigens. Such a non-HLA antibody-mediated acute rejection may be termed AMVR (for “Acute MicroVascular Rejection”).

There also remains a need to develop a method for determining the likelihood of occurrence of such an acute microvascular rejection (AMVR) against a renal allograft in an individual before or shortly after transplantation in order to provide said individuals with a treatment which is specifically adapted and thus avoid a rejection altogether. After transplant, there is also a need to develop a method for detecting the presence of circulating non-HLA anti-ECs Abs as a compagnon test for diagnosing an acute rejection due to non-HLA anti-ECs Abs.

According to the inventors, the identification and characterization of pathogenic anti-endothelial cell Abs (AECAs) would improve our understanding of the mechanisms involved in AMVR and would allow the development of new tools for patient monitoring.

Nevertheless, several hurdles hamper the identification of these AECAs.

First, the development of acute renal dysfunction with histological lesions suggestive of AMR in the absence of anti-HLA DSAs is a relatively rare event. As a consequence, previous studies that were aimed at identifying AECA often included heterogeneous clinical presentations from hyperacute rejection (Dragun et al., Angiotensin II type 1-receptor activating antibodies in renal-allograft rejection. N Engl J Med, 352: 558-569, 2005; Jackson et al., Multiple hyperacute rejections in the absence of detectable complement activation in a patient with endothelial cell reactive antibody. Am J Transplant, 12: 1643-1649, 2012; Zou et al., Antibodies against MICA antigens and kidney-transplant rejection. N Engl J Med, 357: 1293-1300, 2007) to chronic allograft dysfunction (Taniguchi et al., Higher risk of kidney graft failure in the presence of anti-angiotensin II type-1 receptor antibodies. Am J Transplant, 13: 2577-2589, 2013) or patients with a positive EC crossmatch independent of any clinical presentation (Zitzner et al., A prospective study evaluating the role of donor-specific anti-endothelial crossmatch (XM-ONE assay) in predicting living donor kidney transplant outcome. Hum Immunol, 74: 1431-1436, 2013).

Second, the identification of deleterious non-HLA Abs is particularly difficult to achieve in long-term patients, as a broad autoantibody response develops over time after transplantation (Gnjatic et al., Seromic analysis of antibody responses in non-small cell lung cancer patients and healthy donors using conformational protein arrays. J Immunol Methods, 341: 50-58, 2009; Porcheray et al., Chronic humoral rejection of human kidney allografts associates with broad autoantibody responses. Transplantation, 89: 1239-1246, 2010).

### SUMMARY OF THE INVENTION

The present invention aims to meet the here-above indicated needs.

According to one of its objects, the present invention relates to an in vitro method for determining the likelihood of occurrence of an acute microvascular rejection (AMVR) against a renal allograft in an individual, comprising the steps of:

a) measuring, in a sample previously collected from the said individual, the levels of antibodies directed against one or more target antigens selected in the group consisting of ZG16B, LMOD1, BMPR1A, MBP, APEX2, CORO2A, CCBE1, EPHA5, TLE4, EV15L, PLEKHA1, TGM2, ERC1, ZBTB14, TMOD2, MAPK1IP1L, TFEB, PFKFB2, EPHB6 and PNMA2,

b) comparing each antibody level measured at step a) with a reference value,

c) determining the likelihood of occurrence of an acute microvascular rejection (AMVR) against a renal allograft in the said individual based on the comparison of step b).

As used herein, an “Acute MicroVascular Rejection” (also termed “AMVR” herein) means an Antibody-Mediated Rejection (“AMR” or “ABMR”) involving the presence of, or alternatively at least partly caused by, anti-endothelial cells antibodies (AECAs) that are not directed against HLA antigens (i.e. non-HLA antibodies).

According to another one of its objects, the present invention relates to an in vitro method for determining the likelihood of occurrence of an acute microvascular rejection (AMVR) against a renal allograft in an individual, comprising the steps of:

a) incubating human glomerular endothelial cells with a sample of an individual under conditions wherein anti-HLA antibodies do not bind to the said human glomerular endothelial cells,

b) measuring the seroreactivity level of the said sample against the said glomerular endothelial cells,

c) comparing the seroreactivity level obtained at step b) with a reference value,

d) determining the likelihood of occurrence of an acute microvascular rejection (AMVR) against a renal allograft in an individual based on the comparison of step c).

According to another one of its objects, the present invention relates to a kit for determining the likelihood of occurrence of an acute microvascular rejection (AMVR) against a renal allograft in an individual comprising:

(i) one or more immobilized target antigens selected in the group consisting of ZG16B, LMOD1, BMPR1A, MBP, APEX2, CORO2A, CCBE1, EPHA5, TLE4, EV15L, PLEKHA1, TGM2, ERC1, ZBTB14, TMOD2, MAPK1IP1L, TFEB, PFKFB2, EPHB6 and PNMA2, and

(ii) means to detect and/or quantify the levels of antibodies directed against the immobilized target antigens in a sample previously collected from the individual.

The present invention also relates to the use of kits according to the invention for determining the likelihood of occurrence of an acute microvascular rejection (AMVR) against a renal allograft in an individual.

According to another one of its objects, the present invention relates to the use of a kit for determining the likelihood of occurrence of an acute microvascular rejection (AMVR) against a renal allograft in an individual, said kit comprising:

(i) one or more immobilized target antigens selected in the group consisting of ZG16B, LMOD1, BMPR1A, MBP, APEX2, CORO2A, CCBE1, EPHA5, TLE4, EV15L, PLEKHA1, TGM2, ERC1, ZBTB14, TMOD2, MAPK1IP1L, TFEB, PFKFB2, EPHB6 and PNMA2, and

(ii) means to detect and/or quantify the levels of antibodies directed against the immobilized target antigens in a sample previously collected from the individual.

According to a further object, the present invention relates to a kit for determining the likelihood of occurrence of an acute microvascular rejection (AMVR) against a renal allograft in an individual comprising:

(i) immobilized human glomerular endothelial cells under conditions wherein anti-HLA antibodies do not bind to the said human glomerular endothelial cells, and

(ii) means to detect and/or quantify the seroreactivity level of a sample previously collected from the individual against the glomerular endothelial cells.

According to another one of its objections, the present invention relates to the use of a kit for determining the likelihood of occurrence of an acute microvascular rejection (AMVR) against a renal allograft in an individual, said kit comprising:

(i) immobilized human glomerular endothelial cells under conditions wherein anti-HLA antibodies do not bind to the said human glomerular endothelial cells, and

(ii) means to detect and/or quantify the seroreactivity level of a sample previously collected from the individual against the glomerular endothelial cells.

According to a particular embodiment, the seroreactivity level is measured against a reference value corresponding to the level of antibodies directed against a target antigen previously measured in renal allograft recipient individuals with no occurrence of AMVR, or against a pool serum of healthy volunteers.

Within the scope of the present invention, the expression “determining the likelihood of occurrence of an acute microvascular rejection (AMVR) against a renal allograft in an individual” is synonym of determining or predicting the risk of occurrence of an AMVR.

Without wishing to be bound by theory, the inventors believe that determining the likelihood of occurrence of an acute microvascular rejection (AMVR) against a renal allograft in an individual consists in determining the risk of acute rejection induced by non-anti-HLA anti-endothelial cell antibodies.

Indeed, the inventors assume that certain non-anti-HLA anti-endothelial cell antibodies may play a contributing role in acute rejections, and as such may be used as prognostic biomarkers of this phenomenon.

As detailed in the examples below, antibodies able to bind the selected target antigens in accordance with the invention allow for determining the likelihood of occurrence of an acute microvascular rejection (AMVR) against a renal allograft in an individual triggered by non-HLA DSAs (donor-specific antibodies).

Advantageously, antibodies able to bind the selected target antigens in accordance with the invention allow to identify cases of early AMVRs of renal allografts in the absence of anti-HLA DSAs.

Antibodies able to bind the selected target antigens in accordance with the invention allow to identify patients with AMVR but no anti-HLA DSAs from patients with both AMR and anti-HLA DSAs.

This invention also relates to a method for treating acute microvascular rejection (AMVR) in an individual who has received or who is likely to receive a renal allograft, comprising the steps of

a) determining the likelihood of occurrence of an acute microvascular rejection (AMVR) against a renal allograft in the said individual by
(1) measuring, in a sample previously collected from the said individual, the levels of antibodies directed against one or more target antigens selected in the group consisting of ZG16B, LMOD1, BMPR1A, MBP, APEX2, CORO2A, CCBE1, EPHA5, TLE4, EV15L, PLEKHA1, TGM2, ERC1, ZBTB14, TMOD2, MAPK1IP1L, TFEB, PFKFB2, EPHB6 and PNMA2;
(2) comparing each antibody level measured at step a) with a reference value;
(3) determining the likelihood of occurrence of an acute microvascular rejection (AMVR) against a renal allograft in the said individual based on the comparison of step (2)

b) selecting the said individual when the said individual has been determined as being likely to develop an acute microvascular rejection (AMVR) at step a);

c) treating the individual selected at step b) with an appropriate therapeutic treatment capable of diminishing the risk of occurrence of acute microvascular rejection (AMVR) or, better still, of avoiding the appearance of acute microvascular rejection (AMVR).

According to a particular embodiment, the method for treating acute microvascular rejection (AMVR) in an individual who has received or who is likely to receive a renal allograft according to the invention, may optionally further comprise in step a), an additional step (4) of obtaining said likelihood of occurrence of an acute microvascular rejection (AMVR) against a renal allograft in the said individual based on the comparison of step (2) with improved efficiency.

In particular, the step (4) may comprise identifying patients with AMVR but no anti-HLA DSAs.

More particularly, the step (4) may comprise identifying patients with AMVR but no anti-HLA DSAs from patients with both AMR and anti-HLA DSAs.

This invention also pertains to a method for treating acute microvascular rejection (AMVR) in an individual who has received or who is likely to receive a renal allograft, comprising the steps of
a) determining the likelihood of occurrence of an acute microvascular rejection (AMVR) against a renal allograft in the said individual by
   (1) incubating human glomerular endothelial cells with a sample of an individual under conditions wherein anti-HLA antibodies do not bind to the said human glomerular endothelial cells;
   (2) measuring the seroreactivity level of the said sample against the said glomerular endothelial cells;
   (3) comparing the seroreactivity level obtained at step (2) with a reference value (4) determining the likelihood of occurrence of an acute microvascular rejection (AMVR) against a renal allograft in the said individual based on the comparison of step (3);
b) selecting the said individual when the said individual has been determined as being likely to develop an acute microvascular rejection (AMVR) at step a);
c) treating the individual selected at step b) with an appropriate therapeutic treatment capable of diminishing the risk of occurrence of acute microvascular rejection (AMVR) or, better still, of avoiding the appearance of acute microvascular rejection (AMVR).

According to a particular embodiment, the method for treating acute microvascular rejection (AMVR) in an individual who has received or who is likely to receive a renal allograft, may optionally further comprise in step a), an additional step (5) of obtaining said likelihood of occurrence of an acute microvascular rejection (AMVR) against a renal allograft in the said individual based on the comparison of step (3) with improved efficiency.

In particular, the step (5) may comprise identifying patients with AMVR but no anti-HLA DSAs.

More particularly, the step (5) may comprise identifying patients with AMVR but no anti-HLA DSAs from patients with both AMR and anti-HLA DSAs

An appropriate therapeutic treatment as referred to above can be chosen from any known treatment currently available and which is usually prescribed to an individual who is at risk for or who suffers from antibody-mediated rejection.

Such treatments are well known to one skilled in the art and include, but are not limited to treatments comprising immunosuppressant drugs, plasma exchanges, immuno-adsorptions, intravenous immunoglobulins, B-cell depleting agents . . . .

The in vitro methods and kits described herein may also be implemented as “companion tests” to improve diagnostic methods and to improve methods of treatment regularly used to cure or prevent acute organ rejection in an individual before or after a renal allograft.

In some embodiments, the in vitro methods and kits described herein provide clinical information that may be used as such, or that may be used additionally to clinical information that is provided by known methods such as the in vitro observation of a biopsy sample previously collected from the grafted individual. Illustratively, the in vitro methods and kits described herein allow completing information relating to a biopsy sample exhibiting lesions typical from the presence of anti-endothelial cells antibodies, and especially allow completing information relating to a biopsy sample exhibiting lesions typical from the presence of anti-endothelial cells antibodies in the absence of anti-HLA AECAs.

Illustratively, the in vitro methods and kits described herein allow determining the presence of non-HLA AECAs in an individual undergoing an acute rejection of an allograft, and especially of a renal allograft, wherein the detection of non-HLA AECAs may permit the medical practitioner to maintain or adapt the therapeutic treatment to be administered to the allografted individual. Adapting an allografted individual treatment encompasses administering to the said individual one or more active ingredients aimed at reducing or blocking the deleterious effects of AECAs, and especially non-HLA AECAs, caused to the grafted organ tissue.

Companion tests are diagnostic tests used as companion to a therapeutic drug to determine its applicability to a specific person. They are co-developed with drugs to aid in selecting or excluding patient groups for treatment with that particular drug on the basis of their biological characteristics that determine responders and non-responders to the therapy. They are developed based on companion biomarkers, biomarkers that prospectively help predict likely response or severe toxicity.

For instance, a strategy of treatment of acute microvascular rejection including an in vitro method according to the invention as a companion test may consist in the following steps:
selecting an individual that is a renal allograft candidate;
transplanting said candidate with a kidney;
determining the likelihood of occurrence of an acute microvascular rejection (AMVR) against a renal allograft in the transplant recipient using an in vitro method according to the invention following the transplant;
treating said transplant recipient with an appropriate therapeutic treatment to avoid an acute microvascular rejection (AMVR) against the renal allograft;
after an appropriate lapse of time has passed and the treatment has had time to have an effect on the recipient, determining once again the likelihood of occurrence of an acute microvascular rejection (AMVR) against a renal allograft in the transplant recipient using an in vitro method according to the invention,
comparing the likelihood of occurrence before and after treatment of the transplant recipient in order to determine whether said treatment has decreased the likelihood of occurrence of an acute microvascular rejection (AMVR), which would suggest that the treatment has been successful.

As used herein, an “individual” or a “patient” considered within the present invention is a mammal, and more preferably an animal of economic importance which encompasses primarily human individuals as well as farms, laboratories or food industries animals, such as sheep, swine, cattle, goats, dogs, cats, horses, poultry, mice, rats. Most preferably, an individual is a human.

Preferably, an individual according to the invention is (i) a candidate individual for a renal allograft or (ii) a recipient of a renal allograft.

Throughout the text, the following abbreviations may be used:

Abs for antibodies;

AECA for anti-endothelial cell antibody;

AMR for antibody-mediated rejection;

AMVR for acute microvascular rejection;

DSA for donor-specific antibodies;

EC for endothelial cell;

HLA for human leukocyte antigen;

KTR for kidney transplant recipient;

Nabs for natural antibodies.

### LEGENDS OF THE FIGURES

FIG. 1: Study design and workflow.

A nationwide survey identified suspected cases of early (<3 months posttransplant) microvascular (g+ptc score ≥3 (glomerulitis+peritubular capillaritis) according to the Banff classification) rejections of a renal allograft.

After centralized Luminex® SAFB assay testing and central reading of the biopsies, 38 cases were retained for two parallel substudies. A case-control histological study (Study #1) addressed the histological characteristics of the 38 acute microvascular rejections compared to 20 cases of early acute antibody-mediated rejection associated with anti-HLA donor-specific antibodies. A case-control biological study (Study #2) was aimed at identifying non-HLA antibodies by several approaches and used pretransplant serum samples from unsensitized kidney transplant recipients who remained stable during the first posttransplant year and were used as controls.

Finally, an integrative analysis of transcriptomic and proteomic data was performed to identify antibodies targeting glomerular cell-specific antigens (Study #3). To this aim, the differential transcriptomic profiles of microvascular glomerular ECs and macrovascular ECs were combined with the global seroreactivity to protein arrays of serum samples collected immediately before transplantation in kidney transplant recipients with AMVR or stable kidney transplant recipients.

FIG. 2: Pathological characteristics of the early acute microvascular rejections.

(A.) Mean (±SEM) values of the elementary lesions assessed using the Banff classification in the biopsy samples at time of acute microvascular rejection in 38 kidney transplant recipients. Abscissa, from left to right: g: glomerulitis; ptc: peritubular capillaritis; v: intimal arteritis, C4d: C4d staining; i: interstitial inflammation, t: tubulitis; cg: glomerular basement membrane double contours; ci: interstitial fibrosis; ct: tubular atrophy; cv: vascular fibrous intimal thickening; ah: arteriolar hyalinosis. (B.) Glomerulitis (g) and peritubular capillaritis (ptc) scores in the 38 individual cases of acute microvascular rejection.

FIG. 3: Assessment of known AECAs.

(A.) Titers of anti-AT-1R and anti-ETAR antibodies in serum samples collected on the day of transplantation from 23 patients with early AMVR without anti-HLA donor-specific antibodies and 10 nonsensitized KTRs who did not experience any rejection during their first year after transplant and were used as controls. P values were determined using the Mann-Whitney test. In each of the groups Anti-ETAR Abs and Anti-AT1R Abs: (i) dots in the left part: AMVR; dots in the right part: Stable. (B.) Assessments of natural polyreactive antibodies were conducted using flow cytometry to detect reactivity to apoptotic cells or using a dissociation-enhanced lanthanide fluoroimmunoassay (DELFIA) to detect reactivity to malondialdehyde (MDA) in 19 patients with AMVR and 8 controls. P values were determined using the Mann Whitney test. In each of the two groups: (i) dots in the left part: AMVR; dots in the right part: Stable. (C.) Correlation between anti-AT-1R and anti-ETAR antibody titers at the time of transplantation. (D.) Correlation between NAbs reactive to MDA and anti-ETAR antibodies at the time of transplantation. (E.) Correlation between NAbs reactive to MDA and anti-AT-1R antibodies at the time of transplantation. (F.) Analysis of the seroreactivity of serum samples from 10 stable patients and 23 patients with AMVR toward 62 non-HLA antigens using single-antigen flow bead assays. The grey density of each box indicates the MFI of the reaction of the sample to an individual antigen. The thresholds for defining a positive reaction of the patients with to each individual antigen were calculated based on the mean MFI of the control group of stable patients. Samples with an MFI less than the mean+3 standard deviations (SD) were classified as negative and samples with an MFI greater than the mean+3 SD were classified as positive. The number of positive samples is provided on the right and the samples that reached the threshold for positivity are indicated with a cross. Light grey box: 500<MFI<1000. Medium grey box: 1000<MFI<3000. Dark grey box: MFI>3000.

FIG. 4: Endothelial cell crossmatch assays. Sera (diluted 1/4) were incubated with endothelial cells (ECs). Antibody binding was detected by fluorescence-labeled anti-human IgG, and the means of the fluorescence intensity (MFIs) was measured by flow cytometry.

(A.) Comparison of the reactivity of sera from healthy volunteers (HV, n=6) and kidney transplant recipients with (n=19) or without (n=10) early acute microvascular rejection (AMVR) without anti-HLA DSA toward unstimulated microvascular ECs. The data shown are the MFI fold increase compared to a pool of AB serum samples used as negative control. The P value is based on a Kruskal-Wallis test. Asterisks depict pairwise group comparisons by means of Dunn's posttest. ***P<0.01; ****P<0.001. (B.) Sera (diluted 1/4) collected on the day of transplantation or at rejection in 4 patients with AMVR without anti-HLA DSAs were incubated with unstimulated microvascular ECs. Representative histograms showing IgG binding are shown; values indicate the geometric means of the fluorescence intensity. (C.) Serial dilutions of sera from patient AMVR #11 or a pool of healthy volunteers were incubated with renal microvascular ECs before the detection of antibody binding using anti-human IgG. Data shown are the geometric means of the fluorescence intensity. Curves: (1) ◯ HV (AB serum pool; ● AMVR #11 at rejection; ▪ AMVR #11 at Day 0. (D. and E.) Sera (diluted 1/4) collected on the day of transplantation in 19 patients with AMVR were incubated with microvascular (D.) or macrovascular (E.) ECs before (unstimulated) or after a 48-h stimulation with TNFα and IFNγ. A pool of AB sera was used as a negative control (CTL). (F.) Sera (diluted 1:4) collected on the day of transplantation in 2 patients with early acute microvascular rejection (AMVR) or a pool of serum samples from healthy volunteers (HV, n=6) were incubated with renal microvascular endothelial cells (ECs) or epithelial cells. Microvascular ECs were used before or after in vitro differentiation. Representative histograms showing IgG binding are shown, and the values indicate the geometric means of the fluorescence intensity.

FIG. 5: Integrative RNAseq-protein array analysis

Clustering and heat map representation of the transcriptomic data from microvascular and macrovascular ECs. Cell samples (n=3 for microvascular ECs and n=5 for macrovascular ECs) are arranged along the x-axis, whereas differentially expressed genes (n=3427) are arranged along the y-axis. The color of each cell reflects the fold change in the expression of each gene.

### DESCRIPTION OF THE INVENTION

To overcome the challenges in the art, the inventors identified, through a nationwide study, kidney transplant recipients (KTRs) without anti-HLA donor-specific antibodies who experienced acute graft dysfunction within the first 3 months after transplantation and showed severe microvascular injury on biopsy (called AMVR).

The inventors reasoned that early AMVR would likely be due to preformed AECAs, facilitating their identification in pre-transplant serum.

As demonstrated in the experimental part below, the inventors' results suggest that preformed antibodies targeting non-HLA antigens expressed on glomerular endothelial cells are associated with early AMVR and that in vitro cell-based assays are needed to improve risk assessment before transplantation.

The inventors have found that individuals developing an acute microvascular rejection (AMVR) after a renal allograft present particular non-HLA antibodies in their serum which may serve as a diagnostic and a prognostic biomarker and thus help diagnose and anticipate the occurrence of this condition.

As detailed in the examples, the inventors identified specific antibodies directed against one or more target antigens selected in the group consisting of ZG16B, LMOD1, BMPR1A, MBP, APEX2, CORO2A, CCBE1, EPHA5, TLE4, EV15L, PLEKHA1, TGM2, ERC1, ZBTB14, TMOD2, MAPK1IP1L, TFEB, PFKFB2, EPHB6 and PNMA2, and in particular in the group consisting of ZG16B, LMOD1, MBP, TGM2 and PLEKHA1, which represent the top most identified antigens recognized in the sera of more than 30% of AMVR patients.

The selected target antigens in accordance with the invention result in positive identification of antibodies in the sera of more than 30% of AMVR patients without anti-HLA DSAs, more particularly in the sera of more than 40%, more than 45%, more than 50%, more than 55%, more than 60%, more than 65%, more than 70%, more than 75%, more than 80%, more than 85% of AMVR patients without anti-HLA DSAs.

As such, the identification and measure, in the sera of individuals in need thereof, of the levels of the antibodies able to bind to the selected target antigens of the invention allow for a more sensitive and reliable diagnosis and can therefore serve to anticipate more accurately the risk of occurrence of AMVR.

The selected target antigens in accordance with the invention, or the antibodies able to bind those antigens, allow for an improved method of diagnosing the likelihood of occurrence of an acute microvascular rejection (AMVR) against a renal allograft in an individual in need thereof, in particular in an individual without anti-HLA DSAs. In particular, the improved method has an improved sensitivity.

The selected target antigens in accordance with the invention, or the antibodies able to bind those antigens, allow for determining the likelihood of occurrence of an acute microvascular rejection (AMVR) against a renal allograft in an individual triggered by non-HLA DSAs (donor-specific antibodies).

Advantageously, the selected target antigens in accordance with the invention, or the antibodies able to bind those antigens, allow to identify cases of early AMVRs of renal allografts in the absence of anti-HLA DSAs.

The selected target antigens in accordance with the invention, or the antibodies able to bind those antigens, allow to identify patients with AMVR but no anti-HLA DSAs from patients with both AMR and anti-HLA DSAs.

Further, the identification of the above-mentioned antibodies and their use in a method according to the invention helps reduce the number of false-negative and/or the number of false-positive results in the diagnosis of individuals who are tested to determine whether they are at risk of developing an AMVR.

According to a first aspect, the present invention relates to an in vitro method for determining the likelihood of occurrence of an acute microvascular rejection (AMVR) against a renal allograft in an individual, comprising the steps of:

a) measuring, in a sample previously collected from the said individual, the levels of antibodies directed against one or more target antigens selected in the group consisting of ZG16B, LMOD1, BMPR1A, MBP, APEX2, CORO2A, CCBE1, EPHA5, TLE4, EV15L, PLEKHA1, TGM2, ERC1, ZBTB14, TMOD2, MAPK1IP1L, TFEB, PFKFB2, EPHB6 and PNMA2,

b) comparing each antibody level measured at step a) with a reference value,

c) determining the likelihood of occurrence of an acute microvascular rejection (AMVR) against a renal allograft in the said individual based on the comparison of step b).

Advantageously, a method of the invention implementing the selected target antigens in accordance with the invention, or the antibodies able to bind those antigens, allows to identify patients with AMVR but no anti-HLA DSAs.

More advantageously, a method of the invention implementing the selected target antigens in accordance with the invention, or the antibodies able to bind those antigens, allows to identify patients with AMVR but no anti-HLA DSAs from patients with both AMR and anti-HLA DSAs.

The in vitro method according to the invention allows to determine the likelihood of occurrence of an acute microvascular rejection (AMVR) against a renal allograft in an individual with an enhanced sensitivity. Preferably, the in vitro method of the invention further allows to reduce the number of false-negative and/or false-positive results in the diagnosis of individuals who are tested to determine whether they are at risk of developing an AMVR.

The human major histocompatibility complex HLA is known to be the most polymorphic genetic system in humans. The biological role of the HLA class I and class II molecules is to present processed peptide antigens.

The HLA system is clinically important as transplantation antigens.

HLA class I molecules are expressed on the surface of almost all nucleated cells. Class II molecules are expressed only on B lymphocytes, antigen-presenting cells (monocytes, macrophages, and dendritic cells), and activated T lymphocytes or other activated cells. In particular, HLA-A, HLA-B, and HLA-DR have long been known as major transplantation antigens.

The principal targets of the humoral immune response to the renal allograft are the highly polymorphic HLA antigens, but studies have also implicated antibodies directed against non-HLA antigens in the process of AMR, called AECAs (Delville M, Charreau B, Rabant M, Legendre C, Anglicheau D. Pathogenesis of non-HLA antibodies in solid organ transplantation: Where do we stand? Hum Immunol. 2016 November; 77(11):1055-1062).

Non-HLA antibodies directed against non-HLA antigens are classified into two main categories: alloantibodies directed against polymorphic antigens that differ between the recipient and donor, and antibodies that recognize self-antigens—autoantibodies.

Target antigens according to the invention are Zymogen Granule Protein 16 B (ZG16B), Leiomodin-1 (LMOD1), Bone morphogenetic protein receptor, type IA (BMPR1A), Myelin basic protein (MBP), APEX nuclease 2 (APEX2), Coronin, actin binding protein 2A (CORO2A), Collagen and calcium-binding EGF domains 1 (CCBE1), EPH receptor A5 (EPHA5), Transducin-like enhancer of split 4 (TLE4), Ecotropic viral integration site 5-like (EV15L), Pleckstrin homology domain-containing family A1 (PLEKHA1), Transglutaminase 2 (TGM2), ELKS/RAB6-interacting/CAST family member 1 (ERC1), Zinc finger and BTB domain containing 14 (ZBTB14), Tropomodulin 2 (TMOD2), Mitogen-activated protein kinase 1 interacting protein 1-like (MAPK1IP1L), Transcription factor EB (TFEB), 6-phosphofructo-2-kinase/fructose-2,6-biphosphatase 2 (PFKFB2), EPH receptor B6 (EPHB6) and Paraneoplastic Ma antigen 2 (PNMA2).

According to a particular embodiment, step a) consists of measuring the levels of antibodies directed against one or more target antigens selected in the group consisting of ZG16B, LMOD1, MBP, TGM2 and PLEKHA1.

Within the scope of the present invention, the expression “one or more target antigens” encompasses 1, 2, 3, 4, 5, 6, 7, 8, 9, 10, 11, 12, 13, 14, 15, 16, 17, 18, 19, or 20 target antigens.

In a particular embodiment of the invention, the levels of antibodies directed against other target antigens than those of the invention may also be measured.

These other target antigens may be chosen from those known in the art as being predictive biomarkers of the likelihood of occurrence of an acute microvascular rejection (AMVR) against a renal allograft in an individual.

By “individual” according to the invention, is understood an individual selected from the group consisting of (i) a candidate individual for a renal allograft and (ii) a recipient of a renal allograft.

The in vitro method according to the invention comprises a step of comparing the antibody level directed against a target antigen measured in step a) with a reference value.

The reference value according to the invention may for example be chosen from the antibody level directed against a target antigen previously measured in individuals who received a renal allograft and who have not been subject to an AMVR or against a pool serum of healthy volunteers.

Preferably, the reference value of step b) is the level of antibodies directed against a target antigen previously measured in renal allograft recipient individuals with no occurrence of AMVR or against a pool serum of healthy volunteers.

A “healthy volunteer” according to the invention, is an individual whose physiological state does not require a kidney transplant.

As such, individuals who suffer from diseases which may require a kidney transplant are not considered as healthy volunteers according to the invention. For example, individuals who suffer from diabetes, chronic glomerulonephritis, polycystic kidney disease, sickle cell nephropathy, high blood pressure, severe defects of the urinary tract, or chronic kidney disease are not considered as healthy volunteers in the context of the invention.

According to the invention, the individual's sample previously collected of step a) is selected in the group consisting of whole blood, blood plasma and blood serum, in particular in the group consisting of blood plasma and blood serum.

According to a further aspect, the invention relates to an in vitro method for determining the likelihood of occurrence of an acute microvascular rejection (AMVR) against a renal allograft in an individual, comprising the steps of:

a) incubating human glomerular endothelial cells with a sample of an individual under conditions wherein anti-HLA antibodies do not bind to the said human glomerular endothelial cells,

b) measuring the seroreactivity level of the said sample against the said glomerular endothelial cells,

c) comparing the seroreactivity level obtained at step b) with a reference value,

d) determining the likelihood of occurrence of an acute microvascular rejection (AMVR) against a renal allograft in the said individual based on the comparison of step c).

This in vitro method consists in an endothelial crossmatch assay.

Advantageously, a method of the invention implementing the selected target antigens in accordance with the invention, or the antibodies able to bind those antigens, allows to identify patients with AMVR but no anti-HLA DSAs.

More advantageously, a method of the invention implementing the selected target antigens in accordance with the invention, or the antibodies able to bind those antigens, allows to identify patients with AMVR but no anti-HLA DSAs from patients with both AMR and anti-HLA DSAs.

The in vitro method according to the invention allows to determine the likelihood of occurrence of an acute microvascular rejection (AMVR) against a renal allograft in an individual with an enhanced sensitivity. Preferably, the in vitro method of the invention further allows to reduce the number of false-negative and/or false-positive results in the diagnosis of individuals who are tested to determine whether they are at risk of developing an AMVR.

Is understood by “seroreactivity”, the presence/appearance of specific antibodies in the sample of an individual.

The seroreactivity according to the invention may be measured using any method known in the art. In particular, use may be made of secondary antibodies which have been previously labeled and which target anti-human IgGs.

The in vitro method according to the invention comprises a step c) of comparing the antibody level directed against a target antigen measured in step b) with a reference value.

The reference value according to the invention may for example be chosen from the antibody level directed against a target antigen previously measured in individuals who have undergone a renal allograft and who have not been subject to an AMVR or against a pool serum of healthy volunteers.

Preferably, the reference value of step c) is the level of antibodies directed against a target antigen previously measured in renal allograft recipient individuals with no occurrence of AMVR or against a pool serum of healthy volunteers.

By “individual” according to the invention, is understood an individual selected from the group consisting of (i) a candidate individual for a renal allograft and (ii) a recipient of a renal allograft.

According to the invention, the individual's sample previously collected of step a) is selected in the group consisting of whole blood, blood plasma and blood serum, preferably in the group consisting of blood plasma and blood serum.

Step a) of the in vitro method according to the invention consists in incubating human glomerular endothelial cells with a sample of an individual.

The glomerulus is a network of capillaries known as a tuft, located at the beginning of a nephron in the kidney.

Preferably, the said human glomerular endothelial cells of the invention consist of a human glomerular endothelial cell line.

According to a preferred embodiment, the glomerular endothelial cells of step a) do not express HLA antigens.

According to another preferred embodiment, the sample used at step a) has been previously depleted in anti-HLA antibodies.

According to another preferred embodiment, the HLA antigens encoding genes of the human glomerular endothelial cell line are inactivated.

Methods

Ethics

The multicenter retrospective study was approved by the French Ministry of Research (CCTIRS #1403 ibis, validated 10 Apr. 2014) and by the Ethics Committee “Ile de France II” of Necker Hospital (IRB registration #: 1072, validated 24 Mar. 2014). Each patient from the present study was asked to provide written informed consent to be enrolled in the study.

Central Histological Reading of Renal Allograft Biopsies

Clinically indicated biopsy specimens were fixed in formalin, acetic acid, and alcohol and embedded in paraffin. Tissue sections were stained with hematoxylin and eosin, Masson trichrome, periodic acid-Schiff reagent, and Jones stain for light microscopy evaluation. C4d immunohistochemical staining was systematically performed (rabbit anti-human monoclonal anti-C4d; 1/200 dilution; CliniSciences). Renal allograft biopsies from patients with AMVR but no anti-HLA DSAs and patients with both AMR and anti-HLA DSAs were classified using the updated Banff classification (Haas et al., Banff 2013 meeting report: inclusion of c4d-negative antibody-mediated rejection and antibody-associated arterial lesions. Am J Transplant, 14: 272-283, 2014; Loupy et al., The Banff 2015 Kidney Meeting Report: Current Challenges in Rejection Classification and Prospects for Adopting Molecular Pathology. Am J Transplant, 17: 28-41, 2017) by two pathologists (MR and JPDVH) who were blinded to the patient inclusion groups.

Donor-Specific Anti-HLA Antibodies

The presence of circulating anti-HLA-A, -B, -Cw, -DR, -DQ, -DP DSAs was retrospectively and centrally performed by AC with the use of single-antigen flow bead assays (One Lambda, Canoga Park, Calif.) on the Luminex® platform (Lefaucheur et al., Preexisting donor-specific HLA antibodies predict outcome in kidney transplantation. J Am Soc Nephrol, 21: 1398-1406, 2010) HLA typing of donors and recipients was performed using DNA typing (Innolipa HLA Typing Kit; Innogenetics).

Anti-MICA, Anti-AT1R and Anti-ETAR Antibody Assessment

The presence of anti-MICA (anti-class I-related chain A) antibodies was retrospectively and centrally performed by ACG with the use of flow bead assays (One Lambda, Canoga Park, Calif.) on the Luminex® platform.

Anti-AT1R and anti-ETAR Abs were measured with dedicated sandwich ELISAs (CellTrend GmbH, Luckenwalde, Germany, distributed by One Lambda) strictly following the manufacturer's recommendations. Briefly, a 1/100 serum dilution was added in duplicate to each microplate well and incubated at 4° C. for 2 h. After the washing steps, the plates were incubated for 1 h with the horseradish-peroxidase-labeled goat anti-human IgG used for detection, before washing, substrate addition, incubation and then reaction blocking. A standard curve allowed the optical density signal measured to be translated into a concentration expressed in units/mL of serum.

Assessment of Natural Antibodies

Natural antibodies (NAbs) levels were assessed using two separate methods as described previously (See et al., Ventricular assist device elicits serum natural IgG that correlates with the development of primary graft dysfunction following heart transplantation. J Heart Lung Transplant, 36: 862-870, 2017).

In brief, IgGs purified from the patient sera were tested for their reactivity to UV-induced apoptotic Jurkat cells by flow cytometry on a BD LSR Fortessa instrument (BD Biosciences). All samples were diluted 1 to 2 and assessed using the same instrument settings in the same experiment. As a second method, an ELISA was used to detect NAbs reactive to the oxidized lipid epitope malondialdehyde (MDA). MDA-modified BSA was generated as previously reported4 and used to coat high-binding 96-well plates (Corning, Kennebunk, Me.).

A time-resolved fluorometry-based dissociation-enhanced lanthanide fluoroimmunoassay (DELFIA) was used as a read-out. In brief, a biotinylated anti-human IgG secondary antibody was used followed by europium-labelled streptavidin for detection. Serum purified IgG were tested at a dilution of 1:10 in this assay.

Non-HLA Antibody Detection

Sera were tested against a panel of 62 non-HLA antigens provided as two single antigen flow bead assays and provided by One Lambda Inc. (Canoga Park, Calif.).

One kit contained 57 antigens, and the other one gathered the 5 collagen-bearing beads, as the washing buffer was different for the two assays.

Non-HLA antigens bound to microbeads were incubated with patient serum samples (20 microL serum for 5 microL beads). After washing, the bead-bound antibodies were detected with an anti-IgG PE-labelled secondary antibody (LS-AB2, One Lambda) and read on a Luminex 200 instrument (Luminex Corporation, TX). Results were expressed as MFI values adjusted for non-specific binding using the following formula: MFI adjusted=MFI (target bead)−MFI (negative control bead). Positive values for each individual non-HLA antigen were calculated based on the mean MFI of the control group. Samples with an MFI value less than mean+3 standard deviations (SD) were classified as negative and samples with an MFI value greater than the mean+3 SD were classified as positive.

Endothelial Cell Crossmatching

Sera were tested with a custom endothelial cell (EC) crossmatch adapted from (Canet et al., Profiling posttransplant circulating antibodies in kidney transplantation using donor endothelial cells. Transplantation, 93: 257-264, 2012) using banked primary macrovascular ECs prospectively isolated and stored (DIVAT Sample Biocollection, French Health Ministry project number 02G55) and cultured microvascular ECs (CiGEnC: conditionally immortalized human glomerular ECs) (Satchell et al., Conditionally immortalized human glomerular endothelial cells expressing fenestrations in response to VEGF. Kidney Int, 69: 1633-1640, 2006).

To mimic an inflammatory state, cells were activated by adding inflammatory cytokines (TNF-α and IFN-γ, 100 U/ml, for both, purchased from R&D Systems) to the medium, followed by incubation for 48 h.

After washing with PBS, the cells were trypsinized and washed before incubation with patient sera diluted 1:4 into PBS containing 0.05% BSA for 30 minutes. After two more washings, the cells were incubated with an Alexa Fluor® 488 anti-human IgG antibody (AffiniPure F(ab′)₂ Fragment Donkey Anti-Human IgG (H+L), Interchim) for 20 minutes. Fluorescence was measured by flow cytometry (FACS LSR II®, BD Biosciences), and geometric means of fluorescence intensity were calculated using the FlowJo® software program.

Pooled and individual sera from healthy volunteers with no anti-HLA antibody (Etablissement Français du Sang, Nantes) were used as negative controls. A cut-off of 2.0 for the ratio of geometric mean values for the AMVR patients versus the control group was established to define reactive sera.

RNA Sequencing

Total RNAs were isolated from the CiGEnC cells and from banked primary macrovascular ECs obtained in 5 donors using an RNeasy Kit (Qiagen) including a DNase treatment step. RNA quality was assessed using RNA Screen Tape 6000 Pico LabChips with a Tape Station (Agilent Technologies), and the RNA concentration was measured by spectrophotometry using Xpose (Trinean). RNAseq libraries were prepared starting from 2 μg of total RNA using a TruSeq Stranded mRNA LT Sample Prep Kit (Illumina) as recommended by the manufacturer. Half of the oriented cDNA produced from the poly-A+ fraction was PCR amplified (9 or 10 cycles). The RNAseq libraries were sequenced on an Illumina HiSeq2500 (paired-end sequencing, 130×130 bases, high-throughput mode). On average, 84 million paired-end reads per library sample were produced with a minimum of 47 million reads for one sample. The RNA sequencing data are deposited at European Bioinformatics Institute (Annotare; https://www.ebi.ac.uk/arrayexpress/) under registration number E-MTAB-7003.

Protein Array

ProtoArray™ Human Protein Microarrays v5.1 (Life Technologies, Foster City, Calif.) containing more than 9,000 protein features were used to profile circulating antibodies in 30 Day-0 serum samples including 20 samples of kidney transplant recipients with early AMVR without anti-HLA DSAs and 10 samples of kidney transplant recipients who remained stable over the first posttransplant year (used as controls). The samples were profiled at a 1:500 dilution in singlicate, and a pairwise analysis between the two groups (Group 1 vs. Group 2) was carried out to identify the potential group specificity of the immunogenic antigens. Established protocols (http://www.invitrogen.com) were followed for sample preparation and data acquisition (Mattoon et al., Biomarker discovery using protein microarray technology platforms: antibody-antigen complex profiling. Expert Rev Proteomics, 2: 879-889, 2005; Sboner et al., Robust-linear-model normalization to reduce technical variability in functional protein microarrays. J Proteome Res, 8: 5451-5464, 2009). The data analysis software ProtoArray Prospector 5.2 was used to analyze the signal intensities of fixation.

Statistical Analysis

Protein array data were analyzed by ProtoArray™ Prospector software (Life Technologies). A mean increase in the signal intensity above 2 and a P value below 0.05 were considered significant. For the heat map representation of the protein array data, the normalized average signal of fixation was used.

For RNA sequencing data, FASTQ files were mapped to the ENSEMBL [Human(GRCh38/hg38)] reference using “Hisat2” and counted by “featureCounts” from the “Subread” R package. Read count normalizations and group comparisons were performed by three independent and complementary methods, namely, Deseq2, edgeR, LimmaVoom, and the results of each were compared and grouped. The results were then filtered at P value <0.05 and a fold change of 1.2. Average linkage clustering analysis was implemented in the Cluster 3.0 program and Java Tree View 1.1.6r4 software.

Cluster analysis was performed by hierarchical clustering using the Spearman correlation similarity measure and average linkage algorithm. Heat maps were created with the R package ctc: Cluster and Tree Conversion (http://www.r-project.org/) and imaged by Java Treeview software (Saldanha A J: Java Treeview—extensible visualization of microarray data. Bioinformatics, 20: 3246-3248, 2004) and used to obtain a general overview of the data in terms of the within-array distributions of signals and the between-sample variability.

The R packages “res.pca” and “fviz_pca_ind” were used to process the matched data from the protein array and RNAseq and to perform a PCA.

The overall scoring included the frequency of responses in the AMVR patient group in comparison with the stable patient group and included the relative strength of reactivity observed as previously described (Gnjatic et al., Seromic analysis of antibody responses in non-small cell lung cancer patients and healthy donors using conformational protein arrays. J Immunol Methods, 341: 50-58, 2009).

### EXAMPLES

Kidney transplant recipients (KTRs) were identified through a nationwide survey that was aimed at identifying suspected cases of early AMVRs of renal allografts in the absence of anti-HLA DSAs. Inclusion criteria were first transplantation or retransplantation, a deceased or living donor, acute dysfunction or delayed graft function occurring within the first 3 months posttransplantation, histological features of microvascular inflammation with a g+ptc score according to the Banff classification equal to or above 3, absence of historical or current anti-HLA DSA (A/B/Cw/DR/DQ/DP) assessed by a Luminex® single-antigen bead assay. All biopsies were centrally reassessed, and the absence of anti-HLA DSAs was also centrally confirmed (see Methods above).

Fifty-one KTRs (from 21 centers) with suspected early AMVR in the absence of anti-HLA DSAs (DSA− AMVR) were identified.

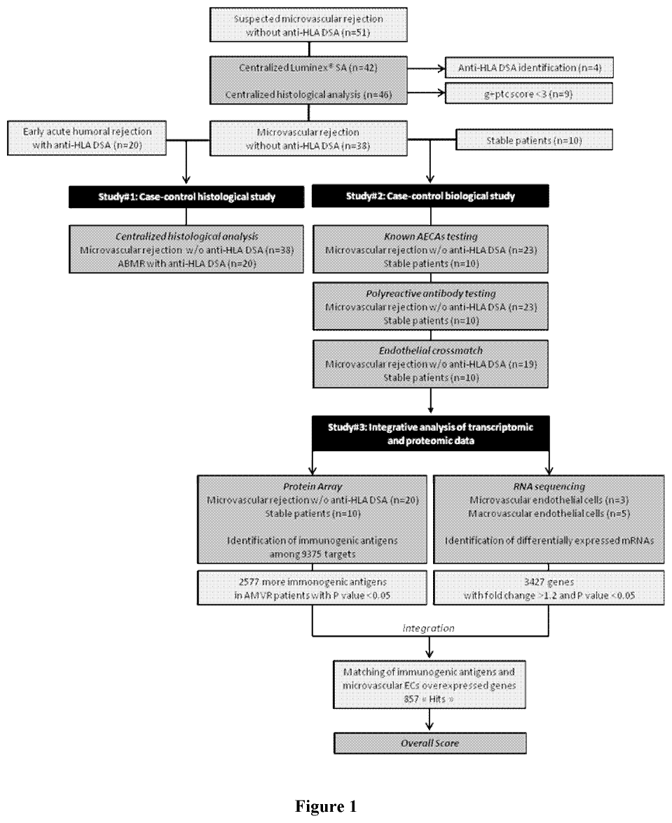

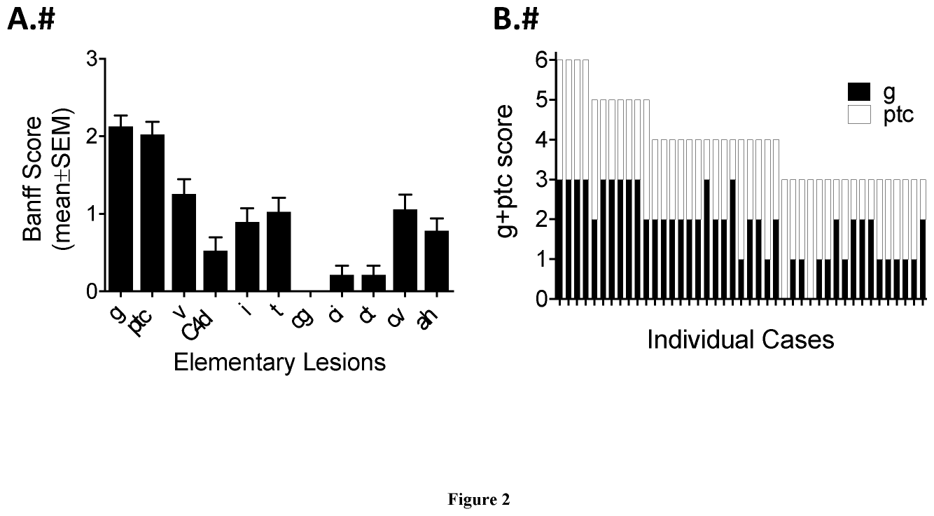

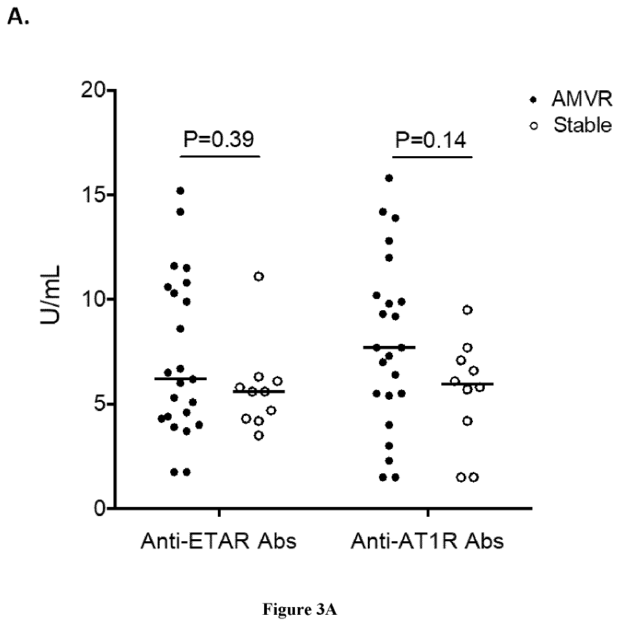

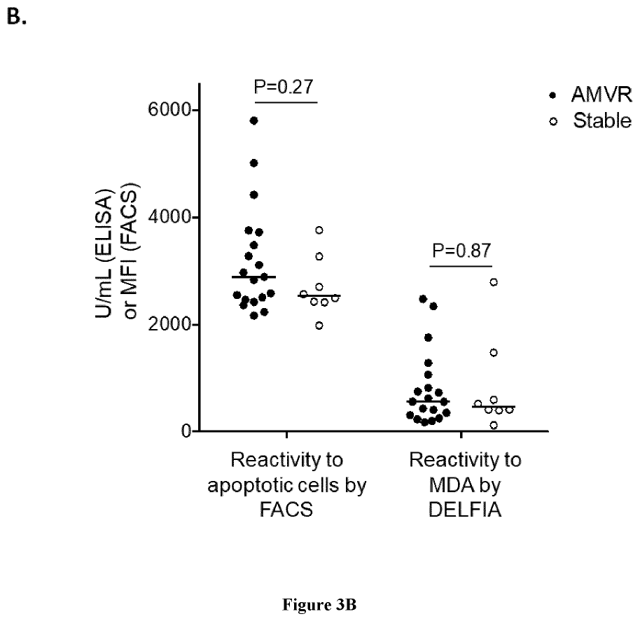

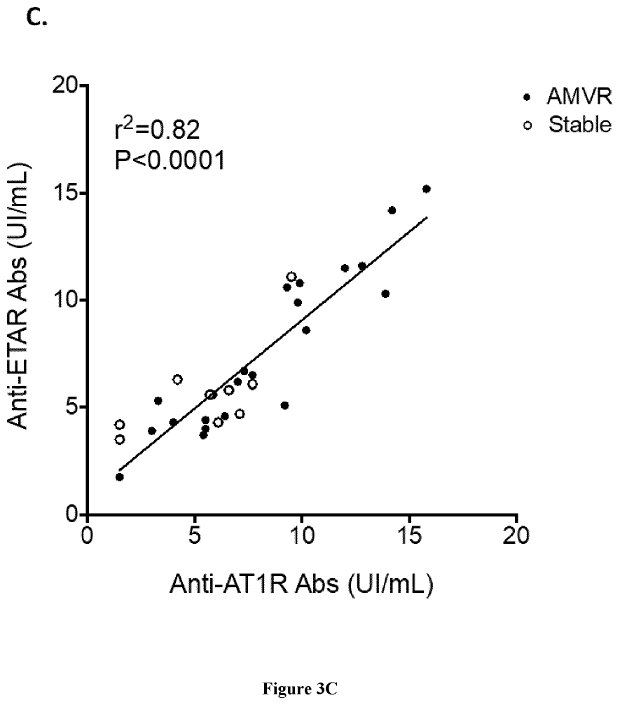

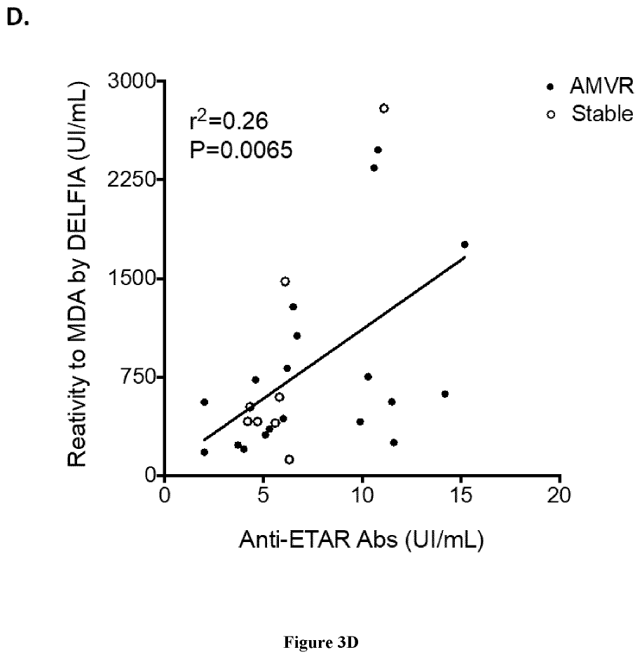

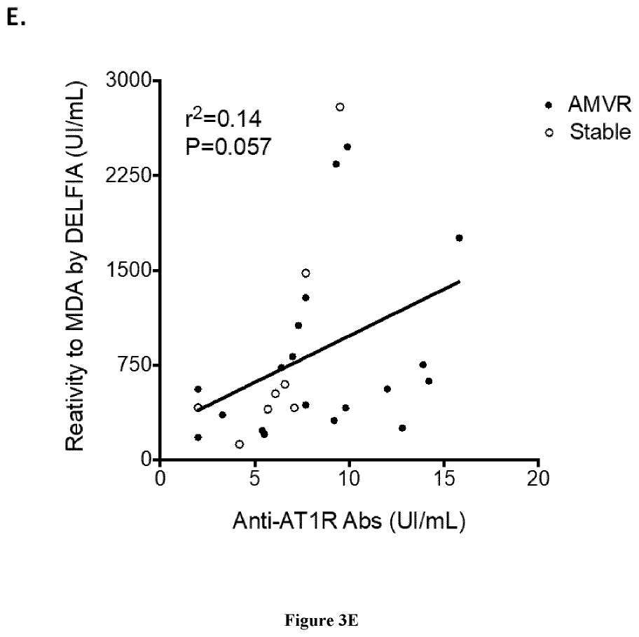

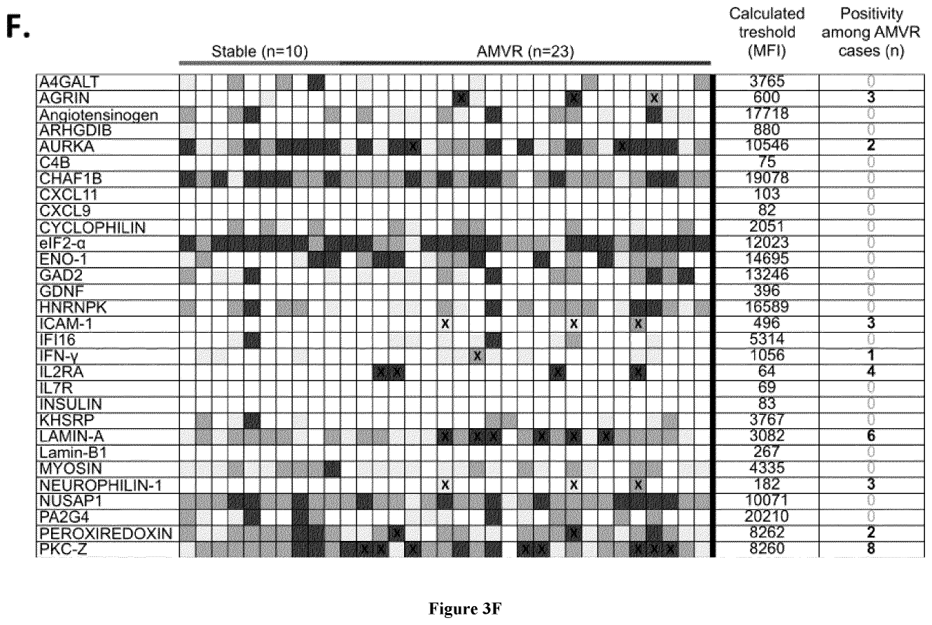

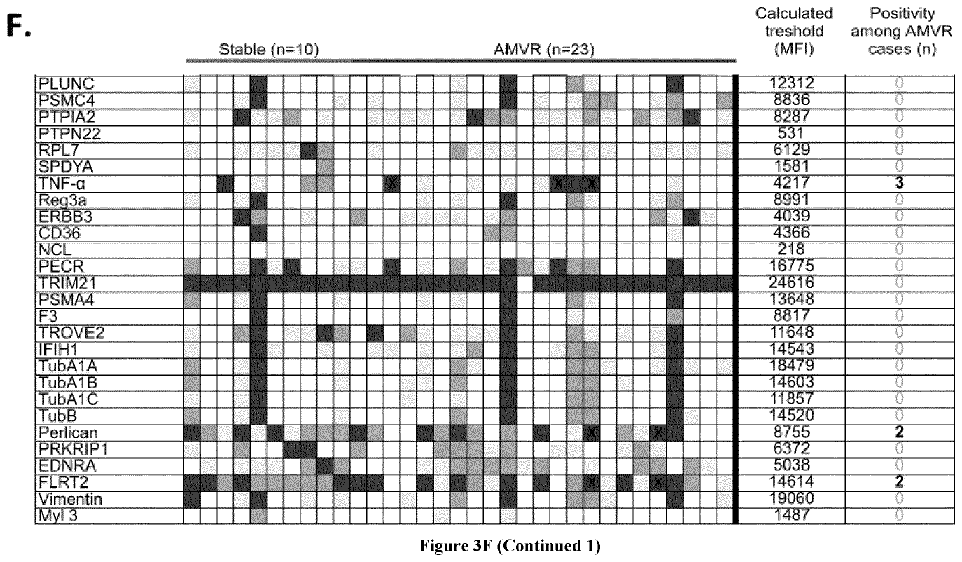

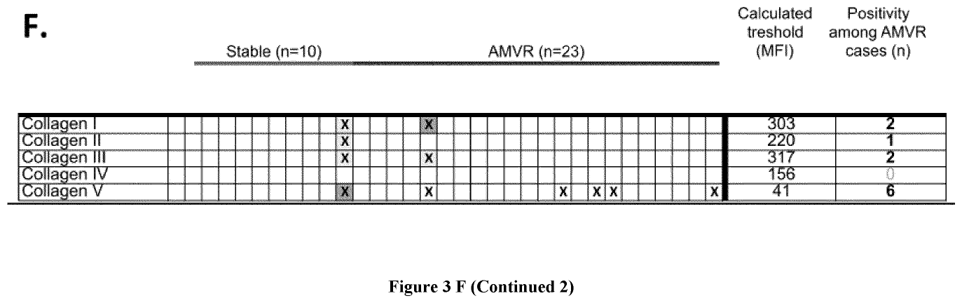

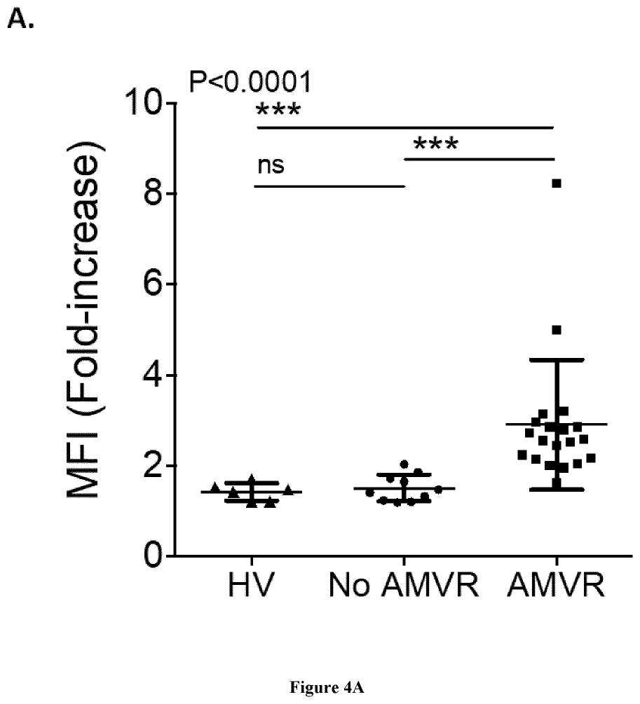

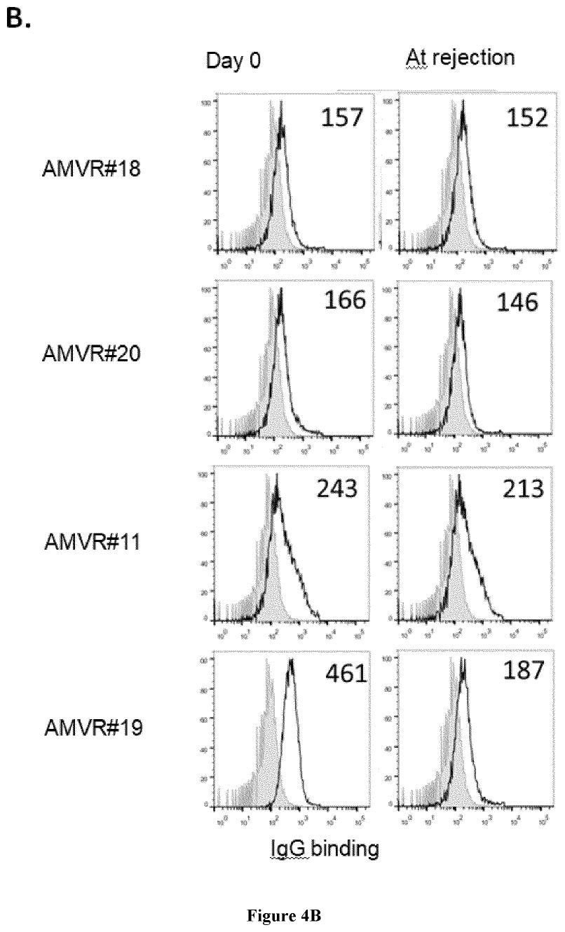

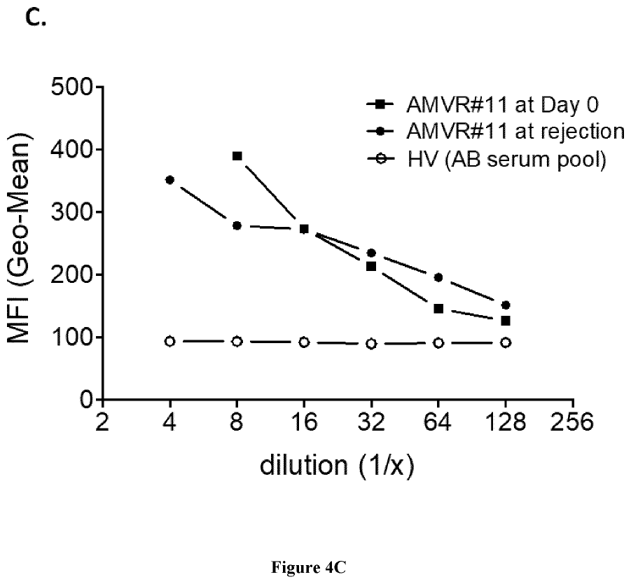

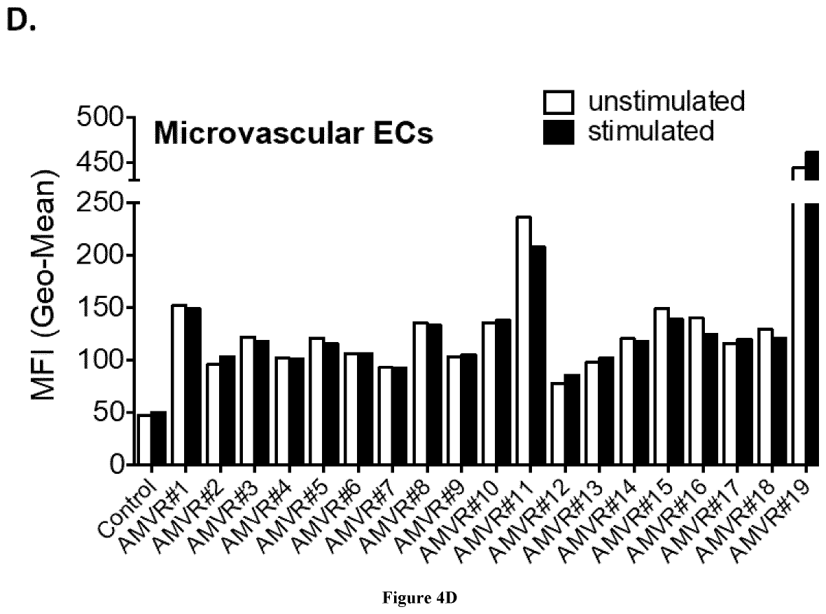

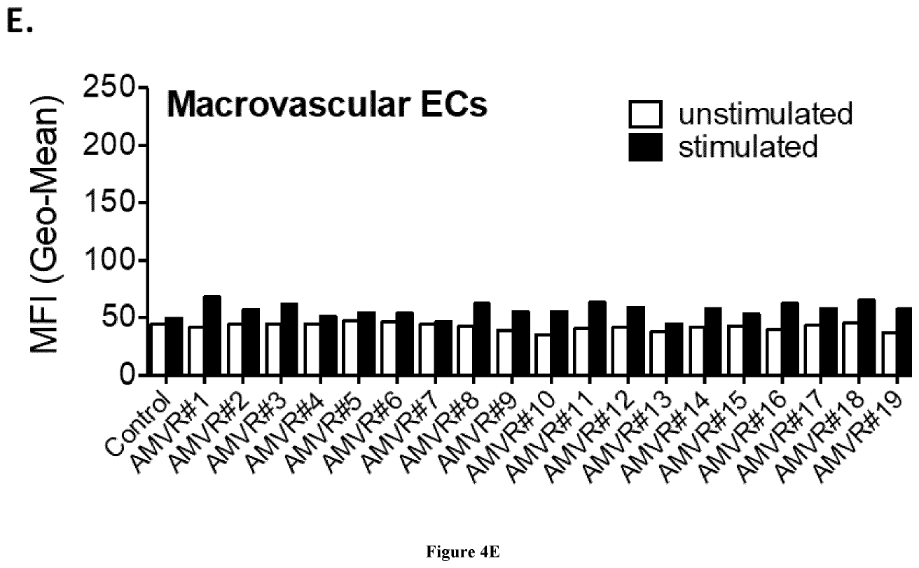

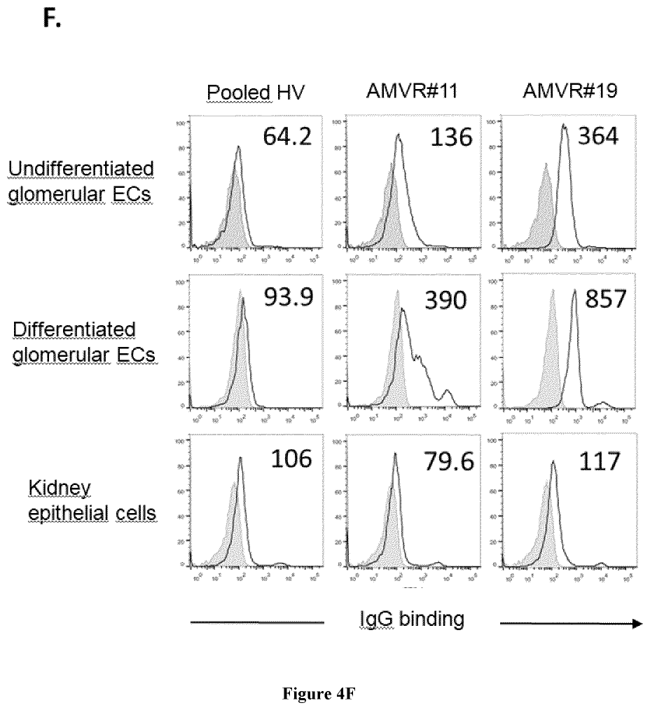

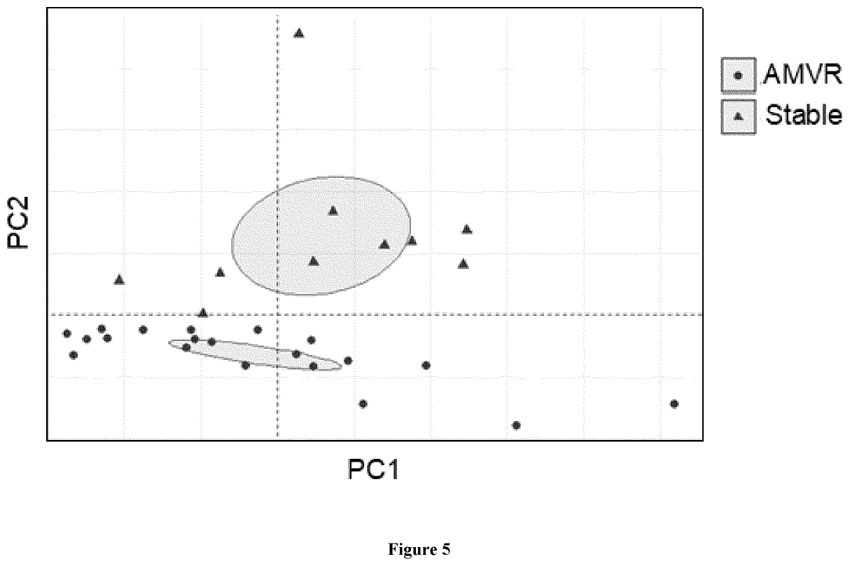

After a central reassessment for anti-HLA DSAs (AC) and a central histological analysis (MR and JPD), the final cohort included 38 patients with confirmed early acute DSA− AMVR (FIG. 1).

For a case-control histological study (FIG. 1), a control group of 20 KTRs with early full-blown AMR with anti-HLA DSAs in the first three months was identified. The patients were matched for age, gender, time of transplantation and immunosuppressive regimen at transplantation.

For a case-control biological study (FIG. 1), a second control group of 10 highly stable patients (i.e., no rejection during the first year) was identified. Patients of this control group were also matched to patients from the AMVR group for age, gender, time of transplantation and immunosuppressive regimen at transplantation.

Patients were 43.0±14.3 years of age (see Table 1 further below). AMVR was diagnosed at a mean time of 11.2±1.7 days for 18 patients still requiring hemodialysis. For the other 20 patients, AMVR was diagnosed because of raising serum creatinine level from 275±187 μmol/L at 15.7±21.4 days to 417±276 μmol/L at 31.8±7.3 days posttransplantation.

The AMVR treatment was heterogeneous. However, rituximab was administered to 31.6% of patients, plasmapheresis to 65.8% and IVIG to 47.4%, suggesting that the patients were considered as having AMR.

A comparison of DSA− AMVR cases with matched DSA+ AMR cases (Table 1) revealed that patients with DSA− AMVR displayed more severe graft dysfunction at 3 months (161±59 μmol/L vs 129±55 μmol/L, P=0.0098) and had numerically increased serum creatinine at 12 months (145±53 μmol/L vs 125±41 μmol/L, P=0.08). Consistent with severe graft injury, proteinuria was common in both groups and after a similar follow-up, the proteinuria in the AMVR cohort was similar to that in the AMR cohort (1.27±1.7 g/g vs 1.0±1.4 g/g, P=0.44).

The central histological reading of the DSA-AMVR cases showed severe microvascular inflammation with a mean g+ptc score of 3.9±0.25 (FIGS. 2A and 2B) and severe endothelial/vascular injury (FIG. 2C-H). Vasculitis was present in 60.5% of cases, and thrombotic microangiopathy and interstitial hemorrhages were observed in 15.8% and 31.6% of cases, respectively (see Table 3 further below).

Compared to DSA+AMR biopsies, DSA− AMVR biopsies demonstrated more severe endothelial/vascular injury with significantly more v lesions (1.3±1.1 vs 0.3±0.8, P=0.001) and numerically more thrombotic microangiopathy (15.8% vs 0%, P=0.08) (Table 2). Compared to patients with AMR, patients in the AMVR group showed significantly more interstitial infiltrates. Overall, T cell-mediated rejection definition according to the Banff classification was not significantly different between the two groups (31.5% vs 10.0%, P=0.18).

The results are presented as the means±SD for continuous variables unless otherwise specified. Frequencies of categorical variables are presented as numbers and percentages. Analyses were performed with GraphPad Prism (version 5.00; GraphPad Software, San Diego, Calif.). For statistical comparisons of the clinical data between two groups, we used unpaired two-tailed t tests and a chi-square test. For statistical comparisons of the in vitro data, we used nonparametric tests. P values <0.05 were considered significant (see methods above).

### Example 1: Non-HLA Antibody Detection

The presence of previously proposed AECAs (Delville et al., Pathogenesis of non-HLA antibodies in solid organ transplantation: Where do we stand? Hum Immunol, 77: 1055-1062, 2016; Gareau et al., Pre-transplant AT1R antibodies correlate with early allograft rejection. Transpl Immunol, 46: 29-35, 2018) was assessed in available serum samples collected at the time of transplant (Day-0), corresponding to a mean time of 22.0±26.2 days prior to the AMVR diagnosis, in 23 patients with early AMVR and 10 stable KTRs used as controls (see Table 2 further below).

Anti-MICA Abs were Detected in Only Two Patients with AMVR.

Titers of angiotensin type 1 receptor (AT1R) and endothelin-1 type A (ETAR) Abs were similar in both groups (FIG. 3A). Regarding AT1R Abs, the inventors did not observe any positivity in the AMVR group or in the stable group (FIG. 3A) using a threshold of 17 UI/mL as proposed by Honger et al. (Human pregnancy and generation of anti-angiotensin receptor and anti-perlecan antibodies. Transpl Int, 27: 467-474, 2014). When the positive threshold of 10 UI/mL proposed by Dragun et al. (Angiotensin II type 1-receptor activating antibodies in renal-allograft rejection. N Engl J Med, 352: 558-569, 2005) was used, 6 AMVR patients were positive for AT1R Abs compared to no patients in the stable group (P=0.14). However, the inventors observed a good correlation between ETAR and AT1R levels with an r2 above 0.8 (P<0.0001), suggesting spreading of the Ab response toward more autoreactivity (FIG. 3B).

IgG natural polyreactive antibody (NAb) levels were assessed in AMVR and control serum samples using two separate methods. No difference in IgG NAbs was observed between the two groups with either method (FIG. 3C). However, as reported in FIG. 3D, the level of IgG NAbs measured by ELISA was significantly correlated with the level of anti-ETAR Abs, supporting the view of a broad autoimmune component.

Sera were also tested against a panel of 62 non-HLA antigens (FIG. 3E). At the time of transplant, 19/23 (83%) AMVR cases had a positive test for at least one of the non-HLA antigens tested.

In total, 16 out of the 62 antigens were positive in at least one AMVR patient.

A total of 45 antigens were found positive, with a maximum of 8 AMVR cases involved in positivity for the protein kinase C.

### Example 2: Endothelial Cell Crossmatch

An EC crossmatch assay was developed to assess serum reactivity to human microvascular glomerular ECs (Satchell et al., Conditionally immortalized human glomerular endothelial cells expressing fenestrations in response to VEGF. Kidney Int, 69: 1633-1640, 2006). As ECs express class I and class II HLA antigens, this analysis was restricted to AMVR patients, stable KTRs or healthy volunteers with no circulating anti-HLA Abs to avoid any HLA-dependent cell reactivity.

Strikingly, the seroreactivity against glomerular ECs was significantly increased in AMVR sera (FIG. 4A), whereas limited reactivity was observed in healthy volunteers (n=6) or stable KTRs (n=10). Seroreactivity against non-HLA antigens was only due to IgG, as no IgM reactivity was observed (data not shown). This IgG reactivity was present at Day-0 (FIG. 4B) and persisted to the time of rejection. Serial titration of positive sera demonstrated high Ab titers (FIG. 4C).

To better characterize this seroreactivity in AMVR patients, crossmatches were also performed in resting ECs and after TNF-α and IFN-γ stimulation.

In healthy controls, even after cell activation, no significant reactivity to glomerular ECs was observed, compared to 89% positivity in AMVR patients. Interestingly, the high-level seroreactivity in AMVR patients was not inflammation-dependent (FIG. 4D). Moreover, no significant reactivity was observed using primary cultures of human macrovascular ECs as targets, even after cell stimulation (FIG. 4E) or using human renal epithelial cells as targets.

Finally, AMVR seroreactivity was higher against fully differentiated glomerular ECs than against undifferentiated ECs (FIG. 4F).

Altogether, these results suggest that the targeted antigens are selectively and constitutively expressed on the cell surface of glomerular ECs.

### Example 3: Integrative cDNA-Protein Array Analysis for Glomerular EC-Specific Immunogenicity

RNAseq was performed to assess the transcriptome differences between microvascular and macrovascular ECs. A protein array was performed on patient serum to assess the seroreactivity of stable KTRs and AMVR patients.

As AMVR seroreactivity specifically targeted glomerular ECs but not macrovascular ECs, the inventors first assessed the differential transcriptomic profiles of these two cell types in order to identify antigens restricted to microvascular ECs (FIG. 5A).

Unsupervised hierarchical clustering of mRNA expression patterns correctly classified the microvascular and the macrovascular ECs (FIG. 5A) suggesting that microvascular glomerular ECs have a distinct transcriptomic profile. Next, read count normalizations and group comparisons were performed by three independent and complementary methods that allowed the identification of 3427 differentially expressed transcripts in the two cell types (FIG. 6), including 2195 genes that are significantly overexpressed in microvascular ECs compared with macrovascular ECs (available online, www.ebi.ac.uk/fg/annotare E-MTAB-7003).

The inventors then used a protein array platform to assess the reactivity of serum samples collected immediately before transplantation from 20 patients with early AMVR and 10 patients who remained stable over the first posttransplant year to approximately 9375 antigens. Evaluation of the average signals for the anti-human IgG were within the expected ranges and were consistent across the arrays, demonstrating the good quality of the samples in both groups. Unsupervised principal component analysis (PCA) demonstrated a clear separation of AMVR patients' sera from stable patients' sera (FIG. 5B) suggesting that the global seroreactivity profile is different in AMVR patients.

Following normalization, individual antigens from protein arrays were ranked according to the frequency of reactivity of AMVR sera compared to that of control sera. To be considered of interest, antigen-specific responses had to be more prevalent in the AMVR patients' sera than in the stable patients, thus possibly representing shared immunogenic events against microvascular ECs. Compared with stable patients' sera, AMVR sera reacted preferentially with 136 of 9375 antigens (unadjusted P<0.05, see Table 2 further below), but with great variability among individuals as illustrated in FIG. 5C.

The inventors next performed an integrative analysis (FIG. 1) combining the serological responses of the AMVR and stable KTRs to the microvascular EC-specific mRNA expression profiles, with the aim of identifying non-HLA Abs in AMVR patients that target proteins specifically expressed by glomerular microvascular ECs. This strategy allowed them to identify a list of 857 matches of immunogenic antigens and overexpressed genes in microvascular ECs (FIG. 1).

Given that seroreactivity was highly variable among AMVR patients, the inventors rank-ordered the 857 potential targets by using a previously described method (Gnjatic et al., Seromic analysis of antibody responses in non-small cell lung cancer patients and healthy donors using conformational protein arrays. J Immunol Methods, 341: 50-58, 2009) that calculates a global score for each candidate by including the frequency of seroreactivity in AMVR patients in comparison with that of stable patients and the relative strength of the reactivity. Altogether, these results suggest that numerous unidentified AECAs are present in AMVR patients but not in stable patients (see Table 4 further below).

Discussion

The concept that AMR may arise in the absence of anti-HLA DSA is universally accepted (Loupy et al., The Banff 2015 Kidney Meeting Report: Current Challenges in Rejection Classification and Prospects for Adopting Molecular Pathology. Am J Transplant, 17: 28-41, 2017). This particular type of rejection is still improperly diagnosed, primarily because of the unknown specificity of the non-HLA Abs associated with its manifestation. Its clinical course and impact on the transplant outcome is also largely unknown. In an effort to better understand this complication, we studied a cohort of highly selected KTRs who experienced an AMR likely triggered by non-HLA DSAs.

Aside from circulating Abs, C4d deposition in peritubular capillaries is considered the best surrogate of antibody-induced injury even if this marker can occasionally be absent in conventional AMR (Haas M, C4d-negative antibody-mediated rejection in renal allografts: evidence for its existence and effect on graft survival. Clin Nephrol, 75: 271-278, 2011; Honger et al., C4d-fixing capability of low-level donor-specific HLA antibodies is not predictive for early antibody-mediated rejection. Transplantation, 89: 1471-1475, 2010) or in the context of suspected AECA-related AMR (Jackson et al., Multiple hyperacute rejections in the absence of detectable complement activation in a patient with endothelial cell reactive antibody. Am J Transplant, 12: 1643-1649, 2012; Dragun et al., Non-HLA-antibodies targeting Angiotensin type 1 receptor and antibody mediated rejection. Hum Immunol, 73: 1282-1286, 2012). In the absence of a consensual definition, the inventors restricted their inclusion criteria to patients with significant microvascular inflammation. In addition, the inventors selected KTRs experiencing acute rejection within the first three months posttransplantation resulting presumably from preformed Abs. These criteria allowed them to identify cases with a homogeneous clinical and pathological presentation. In addition to a severe clinical phenotype, the histological assessment demonstrated a dramatic involvement of the vascular wall with an unusual frequency of “v” lesions, thrombotic microangiopathy and interstitial hemorrhages. Long-term follow-up of these patients, showing allograft dysfunction and glomerular proteinuria were also concordant with an antibody-mediated immune injury.

Numerous AECAs have been reported in the last decade (Delville et al., Pathogenesis of non-HLA antibodies in solid organ transplantation: Where do we stand? Hum Immunol, 77: 1055-1062, 2016). In the present study, the inventors focused on anti-AT1R2, anti-ETAR (Hiemann et al.: Non-HLA antibodies targeting vascular receptors enhance alloimmune response and microvasculopathy after heart transplantation. Transplantation, 94: 919-924, 2012; Banasik et al., The impact of non-HLA antibodies directed against endothelin-1 type A receptors (ETAR) on early renal transplant outcomes. Transpl Immunol, 30: 24-29, 2014) and NAbs (Gao et al., Evidence to Support a Contribution of Polyreactive Antibodies to HLA Serum Reactivity. Transplantation, 100: 217-226, 2016; See et al., Ventricular assist device elicits serum natural IgG that correlates with the development of primary graft dysfunction following heart transplantation. J Heart Lung Transplant, 36: 862-870, 2017). While none of these candidates clearly identified the AMVR patients compared to stable KTRs, the more surprising result was that they were all correlated to each other. Indeed, the inventors found a strong correlation (r2=0.82) between anti-AT1R and anti-ETAR Abs, a finding that was also reported previously in the context of heart (Hiemann et al., Non-HLA antibodies targeting vascular receptors enhance alloimmune response and microvasculopathy after heart transplantation. Transplantation, 94: 919-924, 2012) and renal transplantation (Gareau et al., Pre-transplant AT1R antibodies correlate with early allograft rejection. Transpl Immunol, 46: 29-35, 2018). This observation supports the view that a broad autoimmune response may occur in some patients. In line with this hypothesis, Butte et al. previously identified an autoantibody signature in patients with renal insufficiency compared to controls, thus suggesting that end-stage renal damage may release proteins, not otherwise recognized as self-antigens, leading to an adaptive humoral response (Butte et al.: Protein microarrays discover angiotensinogen and PRKRIP1 as novel targets for autoantibodies in chronic renal disease. Mol Cell Proteomics, 10: M110 000497, 2011). In addition, a longitudinal analysis of the Ab response of pretransplantation and posttransplantation sera through a protein array demonstrated a significant enrichment of Ab response against kidney compartments, again suggesting that chronic organ damage can induce a wide autoantibody response (Gnjatic et al.: Seromic analysis of antibody responses in non-small cell lung cancer patients and healthy donors using conformational protein arrays. J Immunol Methods, 341: 50-58, 2009). Whether this autoimmune response observed in end-stage renal disease patients and transplant recipients is due to the release of self-antigens by the damaged organ or to a systemic B cell deregulation remains unresolved. In this regard, the inventors' observation that the global Ab response before transplantation clearly identified AMVR patients' sera from stable patients' sera supports the hypothesis of systemic B cell deregulation. More recently, an association between endothelial crossmatch positivity and AT1R Abs has also been reported (Philogene et al., Anti-Angiotensin II Type 1 Receptor and Anti-Endothelial Cell Antibodies: A Cross-Sectional Analysis of Pathological Findings in Allograft Biopsies. Transplantation, 101: 608-615, 2017). However, in view of the burst in autoimmunity observed in some patients, this association does not prove causation. Indeed, the findings observed by Dinavahi et al. (Antibodies reactive to non-HLA antigens in transplant glomerulopathy. J Am Soc Nephrol, 22: 1168-1178, 2011) and Porcheray et al. (Chronic humoral rejection of human kidney allografts associates with broad autoantibody responses. Transplantation, 89: 1239-1246, 2010) that autoimmune profiles induced by transplantation are unique to each individual patient also suggest that this response could be the result of systemic B cell deregulation rather than a response to potential cryptic epitopes unmasked during chronic renal injury.

The inventors also evaluated the seroreactivity to a panel of 62 non-HLA antigens provided as two single antigen flow bead assays. If no antigen appeared involved as a positive target in the majority of AMVR cases, 8/23 AMVRs were found to have Abs against protein kinase C, which has been previously associated with acute rejection and graft loss after kidney transplantation (Sutherland et al., Protein microarrays identify antibodies to protein kinase Czeta that are associated with a greater risk of allograft loss in pediatric renal transplant recipients. Kidney Int, 76: 1277-1283, 2009).

If this “candidate gene” approach did not lead to irrefutable candidates, the inventors' crossmatch assay identified preformed IgGs targeting antigens constitutively expressed on glomerular ECs, in a compartment-specific fashion, no response or a minimal response being observed to macrovascular cells, epithelial cells and smooth muscle cells (not shown). This reactivity was highly specific to AMVR patients, thus supporting the inventors' primary hypothesis that AMVR cases are true AMRs.

In an effort to identify the culprits, the inventors profiled the global IgG Ab response in AMVR patients compared to that in controls using protein arrays. The two main conclusions of this “antibodyome-wide” approach were that the global antibodyome correctly classified AMVR cases but that no single specific Ab could explain the disease, even though several Abs that emerged from the combined analysis of transcriptomic and proteomic data have been already reported in the context of autoimmune diseases (see Supplementary discussion below). This finding suggests that patients suffering non-HLA Abs-induced AMRs have profound alterations of their seroreactivity but with little redundancy, and some of their Abs are able to bind to glomerular cells. Altogether, the inventors' observation complements the abovementioned literature and suggests that an attempt to identify a common Ab that may explain the entire spectrum of disease may not succeed.

In conclusion, the inventors addressed the challenging problem of AMR in the absence of anti-HLA Abs in an original way by identifying a highly selected cohort of patients who likely suffered this unusual and difficult-to-diagnose entity. Previously identified non-HLA Abs failed to differentiate AMVR cases from stable patients, but an innovative EC crossmatch identified a universal IgG reactivity to microvascular glomerular ECs. A deep integrative analysis of transcriptomic and proteomic data revealed a large Ab response deregulation with little redundancy among individuals. Altogether, our results suggest that in vitro cell-based assays are needed to assess the presence of EC Abs with a potential deleterious effect after transplantation.

Supplementary Discussion

In this study, the inventors assessed the presence of unknown AECAs in KTRs sera. These unknown AECAs specifically target microvascular endothelial antigens.

The inventors performed an integrative analysis combining the serological responses of AMVR patients and stable KTRs to the microvascular ECs-specific mRNA expression profiles in order to identify antigens of interest. The top most identified antigens were recognized by more than 30% of AMVR patients.

The antigen with the highest score in AMVR patients was ZG16B (Zymogen Granule Protein 16B). It was immunogenic in 90.9% of AMVR patients compared with 33.3% of stable KTRs. Interestingly, ZG16B is a protein identified in urinary exosomes (Prunotto et al., Proteomic analysis of podocyte exosome-enriched fraction from normal human urine. J Proteomics, 82: 193-229, 2013). Exosomes originate as internal vesicles of multivesicular bodies and are released after fusion with the plasma membrane into the extracellular environment. Urinary exosomes, which contain proteins, lipids and RNAs, are produced by podocytes and, potentially, ECs in glomeruli. A recent report showed that the production of some autoantibodies (such as anti-perlecan) that contribute to rejection in organ transplant recipients can be triggered by exosome-like vesicles (Dieude et al., The 20S proteasome core, active within apoptotic exosome-like vesicles, induces autoantibody production and accelerates rejection. Sci Transl Med, 7: 318ra200, 2015).

The second highest top antigen is leiomodin-1 (LMOD1). It was immunogenic in 68% of AMVR patients with twice the cutoff intensity compared with 25% of stable KTRs. Intriguingly, a recent report showed that autoantibodies targeting LMOD1 are more abundantly detected in the sera of patients with nodding syndrome, an autoimmune epileptic disorder, than in unaffected controls. Thus, the inventors showed that anti-LMOD1 antibodies are directly neurotoxic in an in vitro setting (Johnson et al., Nodding syndrome may be an autoimmune reaction to the parasitic worm Onchocerca volvulus. Sci Transl Med, 9, 2017). The potential deleterious impact of anti-LMOD1 antibodies on microvascular ECs could take part in microvascular lesions but remains to be assessed in the kidney transplant context.

The inventors found three other interesting antigens, namely, myelin basic protein (MBP), transglutaminase 2 (TGM2) and pleckstrin homology domain-containing adapter protein (PLEKHA1) that are all associated with the development of autoantibodies in autoimmune diseases. Anti-MBP Abs are deleterious in multiple sclerosis, whereas anti-PLEKHA1 Abs contribute to type 1 diabetes and anti-TGM2 Abs are involved in celiac disease. In multiple sclerosis, an autoimmune neurodegenerative disease leading to destruction of the myelin sheath, the B cell-mediated contribution is important (Archelos et al., The role of B cells and autoantibodies in multiple sclerosis. Ann Neurol, 47: 694-706, 2000). Thus, autoantibodies targeting MBP have been proposed as biomarkers for clinical prognosis (Berger et al., Antimyelin antibodies as a predictor of clinically definite multiple sclerosis after a first demyelinating event. N Engl J Med, 349: 139-145, 2003). Moreover, anti-MBP Abs were also detected in a murine model of multiple sclerosis (Fritz et al., Induction of experimental allergic encephalomyelitis in PL/J and (SJL/J×PL/J)F1 mice by myelin basic protein and its peptides: localization of a second encephalitogenic determinant. J Immunol, 130: 191-194, 1983). In type 1 diabetes (TD1), although the genes in the HLA region constitute the most important genetic risk, other non-HLA genes also contribute to the development of autoantibodies. Thus, Sharma and colleagues (Identification of non-HLA genes associated with development of islet autoimmunity and type 1 diabetes in the prospective TEDDY cohort. J Autoimmun, 2018) recently discovered that the PLEAKHA1 region presents a single nucleotide polymorphism (SNP) and is highly associated with T1D. In celiac disease, a long-term autoimmune disorder primarily affecting the small intestine, IgA antibodies targeting the endomysium are autoantigens that play a major role in the pathogenesis of the disease. Interestingly, Dieterich and colleagues identified tissue TGM2 as the endomysial autoantigen¹⁸ (Dieterich et al., Identification of tissue transglutaminase as the autoantigen of celiac disease. Nat Med, 3: 797-801, 1997).

In conclusion, in this study, the inventors developed a homemade endothelial crossmatch assay and identified a common IgG response in AMVR patients' sera that is specifically directed against constitutively expressed antigens of microvascular glomerular cells.

Protein arrays and RNA sequencing were used to identify 857 antigenic targets of these AECAs. Developing an ELISA for routine testing for each of these AECAs is not a conceivable solution, and thus in vitro cell-based assays are needed to assess the presence of AECAs.

Finally, several of these AECAs are already known as autoantibodies involved in autoimmune disorders, suggesting a potential direct effect of AECAs in microvascular injury.

TABLE 1
Patient demographics

| | AMVR without anti-HLA | AMVR with anti-HLA | |
|---|---|---|---|
| Variables | DSA, N = 38 | DSA, N = 20 | P |
| Recipient characteristics | | | |
| Male, n (%) | 25 (65.8) | 13 (65.0) | 1.00 |
| Age at transplantation, mean ± SD, yr | 43.0 ± 14.3 | 50.4 ± 15.9 | 0.11 |
| Cause of end-stage renal disease, n (%) | | | |
| Glomerulonephritis | 10 (26.3) | 4 (20.0) | 0.75 |
| Diabetes | 6 (15.8) | 5 (25.0) | 0.49 |
| Cystic/hereditary/congenital | 7 (18.4) | 3 (15.0) | 1.00 |
| Secondary glomerulonephritis | 3 (7.9) | 2 (10.0) | 1.00 |
| Hypertension | 2 (5.3) | 0 (0.0) | 0.54 |
| Interstitial nephritis | 3 (7.9) | 2 (10.0) | 1.00 |
| Miscellaneous conditions | 2 (5.4) | 3 (15.0) | 0.33 |
| Etiology uncertain | 5 (13.2) | 1 (5.0) | 0.65 |
| Time of dialysis before transplantation, mean ± SD, yr | 3.944.4 | 4.8 ± 4.9 | 0.44 |
| Previous transplantation, n (%) | 11 (28.9) | 3 (15.0) | 0.34 |
| Transplant variables | | | |
| Donor age, mean ± SD, yr | 50.4 ± 12.6 | 52.3 ± 17.4 | 0.93 |
| Deceased donor, n (%) | 28 (73.7) | 17 (85.0) | 0.51 |
| Male donor, n (%) | 17 (44.7) | 8 (40.0) | 0.79 |
| Cold ischemia time, mean ± SD, hr | 15.9 ± 10.4 | 20.5 ± 9.7 | 0.13 |
| Preformed anti-HLA abs with MFI > 500, n (%) | 19 (50.0) | 20 (100.0) | <0.0001 |
| Delayed graft function, n (%) | 18 (47.3) | 7 (35.0) | 0.41 |
| Number of post-transplant hemodialysis session, mean ± SD | 2.5 ± 4.2 | 2.4 ± 2.9 | 0.39 |
| immunosuppressive protocol | | | |
| Induction therapy, n (%) | 38 (100.0) | 19 (95.0) | 0.34 |
| Basiliximab/Thymoglobuline®, n (%) | 33 (86.8)/5 (13.2) | 14 (75.0) / 5 (25.0) | 0.28 |
| Calcineurin inhibitor-based therapy, n (%) | 37 (97.4) | 20 (100.0) | 1.0 |
| Cyclosporine/Tacrolimus, n (%) | 11 (28.9)/26 (68.4) | 3 (15.0)/17 (85.0) | 0.34 |
| Purine synthesis inhibitor, n (%) | 37 (93.9) | 19 (95.0) | 0.35 |
| mTOR-inhibitor, n (%) | 0 (0.0) | 1 (5.0) | 0.35 |
| Steroid, n (%) | 37 (97.4) | 20 (100.0) | 1.0 |
| Acute rejection description | | | |
| Best serum creatinine before AMVR, mean ± SD, umol/L | 275 ± 187 | 195 ± 137 | 0.15 |
| Best Serum creatinine before AMVR, mean ± SD, days | 15.7 ± 21.4 | 8.5 ± 8.2 | 0.64 |
| AMVR diagnosis, mean ± SD, days | 22.0 ± 26.2 | 15.9 ± 13.5 | 0.92 |
| Serum creatinine at rejection, mean ± SD, umol/L | 417 ± 276 | 298 ± 229 | 0.11 |
| Patients on dialysis at time of rejection | 8 (21.1) | 1 (0.05) | 0.14 |
| Acute rejection treatment | | | |
| Steroid, n (%) | 35 (92.1) | 19 (95.0) | 1.00 |
| Thymoglobuline ®, n (%) | 10 (26.0) | 2 (10.0) | 0.19 |
| Rituximab, n (%) | 12 (31.6) | 10 (50.0) | 0.25 |
| Plasmapheresis, n (%) | 25 (65.8) | 15 (75.0) | 0.56 |
| IglV, n (%) | 18 (47.4) | 17 (85.0) | 0.01 |
| Follow-up | | | |
| Serum creatinine at 3 months post-Tx, mean ± SD, umol/L | 161 ± 59 | 129-55 | 0.0098 |
| Serum creatinine at 12 months post-Tx, mean ± SD, umol/L | 145 ± 53 | 125 ± 41 | 0.08 |
| Mean follow-up, mean ± SD, yr | 4.3 ± 3.0 | 3.5 ± 2.7 | 0.25 |
| Serum creatinine at last follow-up, mean ± SD, umol/L | 169-97 | 136 ± 76 | 0.23 |
| Proteinuria³ at last follow-up, mean ± SD, g/g creatinine | 1.27 ± 1.7 (n = 20) | 1.0 ± 1.4 (n = 18) | 0.44 |
| Patient survival at last follow-up, n (%) | 37 (97.3) | 18 (90.0) | 0.12 |
| Graft survival at last follow-up, n (%) | 29 (76.3) | 19 (95.0) | 0.51 |

TABLE 2
Antigens that are more immunogenic in AMVR patients than in stable KTRs (P < 0.05)

| | |
|---|---|
| Percentage | Mean value |

| | Stable | AMVR | Stable | AMVR | | |
|---|---|---|---|---|---|---|
| Protein Locus | Group | Group | Group | Group | P Value | Description |
| BC001135.1 | 8.33% | 45.46% | 1396.80531 | 1723.21153 | 0.01173958 | transient receptor potential cation channel, |
| | | | | | | subfamily M, member 8 (TRPM8) |
| BC001755.1 | 25% | 68.18% | 8417.42279 | 16045.3644 | 0.01309345 | Leiomodin-1 |
| BC002758.1 | 8.33% | 50% | 1356.8921 | 2030.83847 | 0.00614931 | adenosine deaminase, tRNA-specific 1 (ADAT1) |
| BC002955.1 | 41.67% | 77.27% | 2757.81706 | 3461.10623 | 0.03870862 | ubiquitin specific peptidase 2 (USP2) |
| BC003398.1 | 8.33% | 45.46% | 1906.91261 | 3120.22664 | 0.01173958 | MOB1, Mps One Binder kinase activator-like IB (yeast) |
| | | | | | | (MOBKIB) |
| BC007102.1 | 8.33% | 36.36% | 1001.27233 | 1246.26502 | 0.0380784 | Cell differentiation protein RCD1 homolog |
| BC008435.1 | 41.67% | 81.82% | 2013.91388 | 3387.37465 | 0.01839011 | peroxiredoxin 3 (PRDX3) |
| BC011600.1 | 33.33% | 95.46% | 35082.1649 | 42801.6293 | 5.89E−05 | cDNA clone IMAGE:3050953, **** WARNING: |
| | | | | | | chimeric clone**** |
| BC011781.2 | 58.33% | 95.46% | 7076.73332 | 4436.9737 | 0.00766284 | chromosome 9 open reading frame 37 (C9orf37) |
| BC012381.1 | 16.67% | 77.27% | 1701.26378 | 2337.33378 | 0.01161727 | Neuropilin and tolloid-like protein 2 |
| BC014020.1 | 58.33% | 95.46% | 12946.5971 | 13466.3071 | 0.00766284 | BAI 1-associated protein 2 (BAIAP2) |
| BC014394.1 | 25% | 63.64% | 4787.05277 | 7693.77217 | 0.02508746 | A.T hook DNA-binding motif-containing protein 1 |
| BC014667.1 | 16.67% | 72.73% | 56985.0598 | 67019.5845 | 0.00109945 | immunoglobulin heavy constant gamma 1 (G1m marker) |
| | | | | | | (IGHGI) |
| BC014975.1 | 25% | 59.09% | 1364.98021 | 1944.52892 | 0.04457771 | family with sequence similarity 136, |
| | | | | | | member A (FAM136A) |
| BC014991.1 | 33.33% | 86.36% | 55973.8119 | 65219.8809 | 0.00165721 | N-methylpurine-DNA glycosylase (MPG) |
| BC016381.1 | 16.67% | 72.73% | 55040.7019 | 64792.9392 | 0.00109945 | immunoglobulin heavy constant mu (IGHM) |
| BC017968.1 | 8.33% | 59.09% | 1267.5451 | 1901.91689 | 0.01116585 | solute carrier family 16, member 10 (aromatic amino |
| | | | | | | acid transporter) (SLC16A10) |
| BC019337.1 | 33.33% | 81.82% | 51446.6321 | 56255.4887 | 0.00484443 | immunoglobulin heavy constant gamma 1 |
| | | | | | | (G1m marker) (IGHG1) |
| BC022362.1 | 33.33% | 86.36% | 53126.5594 | 61125.3437 | 0.00165721 | cDNA clone MGC:23888 IMAGE:4704496, |
| | | | | | | complete cds |
| BCO23144.1 | 16.67% | 54.55% | 2513.12309 | 7976.44762 | 0.02087528 | SWI/SNF-related matrix-associated |
| | | | | | | actin-dependent regulator of |
| | | | | | | chromatin subfamily A member 5 |
| BC025314.1 | 8.33% | 77.27% | 52856.3307 | 63201.3546 | 3.33E−05 | immunoglobulin heavy constant gamma 1 |
| | | | | | | (G1m marker) (IGHG1) |
| BC026038.1 | 16.67% | 72.73% | 54642.5423 | 64325.87 | 0.00109945 | Ig gamma-1 chain C region |
| BC026070.2 | 16.67% | 50% | 1010.45279 | 2885.35209 | 0.03689584 | tubby like protein 2 (TULP2) |
| BC030814.1 | 8.33% | 90.91% | 26297.2838 | 32756.25 | 3.66E−07 | immunoglobulin kappa variable 1-5 (IGKV1-5) |
| BC032372.1 | 8.33% | 36.36% | 659.660811 | 1569.44368 | 0.0380784 | Ral GEF with PH domain and SH3 binding motif 1 |
| | | | | | | (RALGPS1) |
| BC032416.1 | 8.33% | 36.36% | 910.095187 | 1948.35423 | 0.0380784 | serine/arginine repetitive matrix 2 (SRRM2) |
| BC032451.1 | 16.67% | 77.27% | 47974.5559 | 56698.1655 | 0.00041408 | cDNA clone MGC:40426 IMAGE:5178085, complete cds |
| BC033178.1 | 16.67% | 72.73% | 54148.5147 | 63741.6112 | 0.00109945 | immunoglobulin heavy constant gamma 3 (G3m marker) |
| | | | | | | (IGHG3) |
| BC033689.1 | 25% | 77.27% | 1991.98142 | 2690.84563 | 0.01161727 | MARVEL domain containing 2 (MARVELD2) |
| BC033708.1 | 33.33% | 77.27% | 8233.78203 | 5686.0683 | 0.01161727 | Ral GEF with PH domain and SH3 binding motif 1 |
| | | | | | | (RALGPS1) |
| BC033766.1 | 8.33% | 36.36% | 3128.99802 | 4104.96661 | 0.0380784 | NADH dehydrogenase (ubiquinone) flavoprotein 3, |
| | | | | | | 10 kDa (NDUFV3) |
| BC036184.1 | 16.67% | 50% | 2042.66279 | 2597.21201 | 0.03689584 | Tropomodulin-2 |
| BC036767.1 | 25% | 63.64% | 2701.26286 | 3426.03477 | 0.02508746 | RIB43 A domain with coiled-coils 1 (RIBCI) |
| BC037854.1 | 8.33% | 36.36% | 624.348659 | 1294.67744 | 0.0380784 | dynein, cytoplasmic 1, intermediate chain 1 (DYNC1I1) |
| BC039895.1 | 16.67% | 54.55% | 4798.84974 | 8691.82814 | 0.02087528 | breast cancer anti-estrogen resistance 3 (BCAR3) |
| BC042193.1 | 16.67% | 50% | 1707.73531 | 2311.19213 | 0.03689584 | G patch domain containing 2 (GPATCH2) |
| BC047536.1 | 16.67% | 54.55% | 28924.8231 | 35195.0396 | 0.02087528 | sciellin (SCEL) |
| BC048299.1 | 41.67% | 77.27% | 5120.44732 | 8973.86743 | 0.03870862 | spermatogenesis associated, serine-rich 2 (SPATS2) |
| BC051733.1 | 33.33% | 72.73% | 2679.48847 | 3934.83874 | 0.02416484 | Leucine zipper protein 1, mRNA (cDNA clone |
| | | | | | | MGC:51018 IMAGE:4838475), complete cds |
| BC053984.1 | 33.33% | 77.27% | 55974.4539 | 60117.9603 | 0.01161727 | immunoglobulin heavy variable 4-31 (IGHV4-31) |
| BC054893.1 | 8.33% | 72.73% | 8848.6302 | 10373.7716 | 1.00E−04 | immunoglobulin lambda variable 2-14 (IGLV2-14) |
| BC056508.1 | 8.33% | 36.36% | 915.224054 | 1208.37966 | 0.0380784 | variable charge, Y-linked IB (VCY) |
| BC059405.1 | 33.33% | 68.18% | 3334.35655 | 3249.13179 | 0.04507746 | Transducin-like enhancer protein 4 |
| BC059947.1 | 33.33% | 77.27% | 6843.28379 | 9489.84091 | 0.01161727 | chondrosarcoma associated gene 1 (CSAG1) |
| BC062336.1 | 66.67% | 95.46% | 53450.5972 | 57808.4919 | 0.02955665 | Immunoglobulin heavy constant gamma 1 |
| | | | | | | (Glm marker), mRNA |
| | | | | | | (cDNA clone MGC:71315 IMAGE:6300554), |
| | | | | | | complete cds |
| BC062732.1 | 16.67% | 72.73% | 53095.6339 | 62502.2843 | 0.00109945 | ig kappa chain C region |
| BC066642.1 | 16.67% | 72.73% | 49214.3539 | 57543.3265 | 0.00109945 | Immunoglobulin heavy constant gamma 1 |
| | | | | | | (Glm marker), mRNA |
| | | | | | | (cDNA clone MGC:71306 IMAGE:5451018), |
| | | | | | | complete cds |
| BC067091.1 | 33.33% | 72.73% | 51378.4987 | 54052.6298 | 0.02416484 | Immunoglobulin heavy constant gamma 1 |
| | | | | | | (Glm marker), mRNA |
| | | | | | | (cDNA clone MGC:71316 IMAGE:6301214), |
| | | | | | | complete cds |
| BC067226.1 | 16.67% | 68.18% | 24404.4005 | 27781.4327 | 0.0025987 | Immunoglobulin kappa constant, mRNA (cDNA clone |
| | | | | | | MGC:72070 IMAGE:30349629), complete cds |
| BC069020.1 | 8.33% | 81.82% | 25327.3334 | 31315.2871 | 9.52E−06 | Immunoglobulin heavy constant gamma 1 |
| | | | | | | (Glm marker), mRNA |
| | | | | | | (cDNA clone MGC:78608 IMAGE:6214622), |
| | | | | | | complete cds |
| BC070361.1 | 8.33% | 50% | 34591.1579 | 38146.3129 | 0.00614931 | Immunoglobulin kappa constant, mRNA (cDNA clone |
| | | | | | | MGC:88369 IMAGE:30352586), complete cds |
| BC072419.1 | 66.67% | 95.46% | 34694.5369 | 36192.2431 | 0.02955665 | ig gamma-1 chain C region |
| BC073782.1 | 16.67% | 72.73% | 53097.2245 | 62296.6046 | 0.00109945 | cDNA clone MGC:88796 IMAGE:6295732, |
| | | | | | | complete cds |
| BC073793.1 | 33.33% | 77.27% | 13061.0668 | 13660.5893 | 0.01161727 | cDNA clone MGC:88813 IMAGE:6302307, |
| | | | | | | complete cds |
| BC073937.1 | 33.33% | 81.82% | 7218.87476 | 8315.32892 | 0.00484443 | Immunoglobulin kappa constant, mRNA (cDNA clone |
| | | | | | | MGC:90448 IMAGE:5226105), complete cds |
| BC078670.1 | 33.33% | 72.73% | 31705.0886 | 35052.314 | 0.02416484 | Immunoglobulin heavy constant gamma 1 |
| | | | | | | (Glm marker), mRNA |
| | | | | | | (cDNA clone MGC:88797 IMAGE:6295788), |
| | | | | | | complete cds |
| BC092518.1 | 8.33% | 90.91% | 51483.6881 | 56383.912 | 0.03124079 | ig gamma-1 chain C region |
| BC095489.1 | 16.67% | 54.55% | 17284.7314 | 18949.645 | 0.02087528 | Immunoglobulin kappa constant, mRNA |
| | | | | | | (cDNA clone MGC: |
| | | | | | | 111575 IMAGE:30328747), complete cds |
| BC096272.2 | 33.33% | 77.27% | 10256.9597 | 17416.5038 | 0.01161727 | HIV-1 Rev binding protein, mRNA (cDNA clone |
| | | | | | | MGC: 116938 IMAGE:40006445), complete cds |
| BC099907.1 | 25% | 77.27% | 55249.1599 | 62741.8482 | 0.00265472 | General transcription factor 11-1 |
| IGFBP6_ | 33.33% | 68.18% | 4122.08981 | 4804.45222 | 0.04507746 | IGFBP6 Recombinant Human Protein |
| Recombinant | | | | | | |
| NM_000593.5 | 8.33% | 45.46% | 528.271488 | 1105.95158 | 0.01173958 | transporter 1, ATP-binding cassette, sub-family B |
| | | | | | | (MDR/TAP) (TAPI) |
| NM_001025100.1 | 25% | 63.64% | 1665.66335 | 3239.32664 | 0.02508746 | Myelin basic protein |
| NM_001032293.1 | 16.67% | 54.55% | 17717.1465 | 30063.6859 | 0.02087528 | zinc finger protein 207 (ZNF207), transcript variant 2 |
| NM_001312.2 | 33.33% | 68.18% | 6439.58585 | 9350.98666 | 0.04507746 | cysteine-rich protein 2 (CRIP2) |
| NM 001860.1 | 8.33% | 36.36% | 773.47464 | 1055.78263 | 0.0380784 | solute carrier family 31 (copper transporters), |
| | | | | | | member 2 (SLC31A2) |
| NM_001983.1 | 8.33% | 40.91% | 1162.27207 | 2030.45446 | 0.02152257 | excision repair cross-complementing rodent |
| | | | | | | repair deficiency, |
| | | | | | | complementation group 1 (includes overlapping antisense |
| | | | | | | sequence) |
| NM_002103.3 | 41.67% | 77.27% | 3556.46742 | 4017.64711 | 0.03870862 | (ERCC1), transcript variant 2 |
| | | | | | | glycogen synthase 1 (muscle) (GYSI) |
| NM_002625.1 | 8.33% | 36.36% | 1547.23624 | 6489.90305 | 0.0380784 | 6-phosphofructo-2-kinase/fmctose-2,6-biphosphatase 1 |
| NM_002638.1 | 16.67% | 50% | 1369.97487 | 1913.06652 | 0.03689584 | peptidase inhibitor 3, skin-derived (SKALP) (PI3) |
| NM_002904.4 | 58.33% | 95.46% | 30676.7318 | 34898.2601 | 0.00766284 | RD RNA binding protein (RDBP) |
| NM_002945.2 | 33.33% | 72.73% | 27987.4399 | 31306.8207 | 0.02416484 | replication protein Al, 70 kDa (RPA1) |
| NM_004202.1 | 25% | 59.09% | 1265.71994 | 1948.08053 | 0.04457771 | Thymosin beta-4, Y-chromosomal |
| NM_004302 | 33.33% | 77.27% | 63759.8595 | 68998.1365 | 0.01161727 | Acti-Vin Rib Recombinant Human Protein |
| NM_004329 | 16.67% | 72.73% | 48666.1222 | 57290.137 | 0.00109945 | BMPRIA Recombinant Human Protein |
| NM_004450.1 | 8.33% | 36.36% | 1378.07195 | 2440.3009 | 0.0380784 | enhancer of rudimentary homolog (Drosophila) (ERH) |
| NM 004566.1 | 16.67% | 54.55% | 2267.29416 | 7240.49416 | 0.02087528 | 6-phosphofructo-2-kinase/fructose-2,6-biphosphatase 3 |
| | | | | | | (PFKFB3) |
| NM_004987.3 | 8.33% | 45.46% | 67541.4438 | 70292.9132 | 0.01173958 | LIM and senescent cell antigen-like-containing |
| | | | | | | domain protein 1 |
| NM_005510.2 | 50% | 86.36% | 11890.6894 | 12612.3704 | 0.02564103 | dom-3 homolog Z (C. elegans) (DOM3Z) |
| NM_006413.2 | 8.33% | 36.36% | 1657.80522 | 2085.72063 | 0.0380784 | Ribonuclease P protein subunit p30 |
| NM_006790.1 | 8.33% | 40.91% | 9282.31567 | 11029.2684 | 0.02152257 | myotilin (MYOT) |
| NM_006792.2 | 8.33% | 45.46% | 885.325688 | 4555.077 | 0.01173958 | mortality factor 4 (MORF4), mRNA. |
| NM_007099.1 | 8.33% | 40.91% | 1408.56951 | 1826.0404 | 0.02152257 | acid phosphatase 1, soluble (ACPI), transcript variant 2 |
| NM_007162.1 | 8.33% | 40.91% | 1569.44844 | 2517.03843 | 0.02152257 | transcription factor EB (TFEB) |
| NM_013975.1 | 8.33% | 45.46% | 2660.28757 | 4735.6742 | 0.01173958 | ligase III, DNA, ATP-dependent (LIG3), nuclear |
| | | | | | | gene encoding |
| NM_014049.3 | 8.33% | 40.91% | 1748.76616 | 2188.59181 | 0.02152257 | mitochondrial protein, transcript variant alpha |
| | | | | | | acyl-Coenzyme A dehydrogenase family, member 9 |
| | | | | | | (ACAD9) |
| NM_014268.1 | 8.33% | 45.46% | 771.745138 | 1670.59557 | 0.01173958 | microtubule-associated protein, RP/EB family, |
| | | | | | | member 2 (MAPRE2) |
| NM_014481.2 | 25% | 77.27% | 50704.738 | 59300.2919 | 0.00265472 | APEX nuclease (apurinic/apyrimidinic endonuclease) 2 |
| | | | | | | (APEX2), nuclear gene encoding mitochondrial protein |
| NM_014923.2 | 8.33% | 54.55% | 1356.24474 | 2979.84535 | 0.00307465 | fibronectin type III domain containing 3A (FNDC3A), |
| | | | | | | transcript variant 2 |
| NM_016564.1 | 25% | 63.64% | 2048.59549 | 3091.0582 | 0.02508746 | cell cycle exit and neuronal differentiation 1 (CEND1) |
| NM_017451.1 | 33.33% | 81.82% | 9050.21611 | 9367.48708 | 0.00484443 | BAI 1-associated protein 2 (BAIAP2), transcript variant 2 |
| NM_017706.2 | 8.33% | 36.36% | 1153.99859 | 1653.13306 | 0.0380784 | WD repeat-containing protein 55 |
| NM_017735.3 | 16.67% | 54.55% | 746.01178 | 804.122035 | 0.02087528 | Tetratricopeptide repeat protein 27 |
| NM_018047.1 | 25% | 59.09% | 4851.00548 | 4819.61219 | 0.04457771 | Pre-mRNA-splicing factor RBM22 |
| NM_020992.2 | 66.67% | 95.46% | 3536.37724 | 3557.85099 | 0.02955665 | PDZ and LIM domain 1 (elfin) (PDLIM1) |
| NM_022977.1 | 8.33% | 50% | 639.918285 | 1299.29725 | 0.00614931 | acyl-CoA synthetase long-chain family member 4 |
| | | | | | | (ACSL4), transcript variant 2 |
| NM 031469.1 | 8.33% | 40.91% | 733.26459 | 1623.4096 | 0.02152257 | SH3 domain binding glutamic acid-rich protein like 2 |
| | | | | | | (SH3BGRL2) |
| NM_032975.2 | 8.33% | 40.91% | 1654.98553 | 2778.24497 | 0.02152257 | Dystrobrevin, alpha (DTNA), transcript variant 2, mRNA |
| NM_053005.2 | 8.33% | 54.55% | 2815.60617 | 4209.51162 | 0.00307465 | HCCA2 protein (HCCA2) |
| NM_078630.1 | 66.67% | 95.46% | 2858.25416 | 3313.10931 | 0.02955665 | male-specific lethal 3-like 1 (Drosophila) (MSL3L1), |
| | | | | | | transcript variant 2 |
| NM 080548.1 | 8.33% | 36.36% | 3210.77672 | 5727.81597 | 0.0380784 | Tyrosine-protein phosphatase non-receptor type 6 |
| NM_130807.1 | 16.67% | 54.55% | 3780.46781 | 5514.13132 | 0.02087528 | MOB1, Mps One Binder kinase activator-like 2A (yeast) |
| | | | | | | (MOBKL2A) |
| NM_144578.1 | 8.33% | 40.91% | 743.908335 | 1394.74235 | 0.02152257 | chromosome 14 open reading frame 32 (C14orf32) |
| NM 145061.1 | 25% | 59.09% | 1315.65955 | 1739.6403 | 0.04457771 | chromosome 13 open reading frame 3 (C13orf3) |
| NM_145252.1 | 33.33% | 90.91% | 5727.20399 | 7926.82147 | 0.00041774 | similar to common salivary protein 1 (LOC124220) |
| NM_145716.2 | 8.33% | 36.36% | 3698.55666 | 9002.01724 | 0.0380784 | single stranded DNA binding protein 3 (SSBP3), |
| | | | | | | transcript variant 1 |
| NM_173191.2 | 8.33% | 59.09% | 244.098103 | 811.032206 | 0.04457771 | Kv channel interacting protein 2 (KCNIP2), |
| | | | | | | transcript variant 2 |
| NM_173468.2 | 8.33% | 40.91% | 1216.85016 | 1574.34065 | 0.02152257 | MOB1, Mps One Binder kinase activator-like 1A (yeast) |
| | | | | | | (MOBKL1A) |
| NM_175907.3 | 16.67% | 72.73% | 62610.5486 | 73700.5011 | 0.00109945 | zinc binding alcohol dehydrogenase, domain |
| | | | | | | containing 2 (ZADH2) |
| NM_177973.1 | 16.67% | 54.55% | 3737.01917 | 2078.94662 | 0.02087528 | sulfotransferase family, cytosolic, 2B, member 1 |
| | | | | | | (SULT2B1), transcript variant 2 |
| NM_178044.1 | 8.33% | 40.91% | 637.761772 | 889.252908 | 0.02152257 | GIY-YIG domain containing 2 (GIYD2), |
| | | | | | | transcript variant 2 |
| NM_178553.2 | 8.33% | 36.36% | 12927.2234 | 19337.4665 | 0.0380784 | Uncharacterized protein C2orf53 |
| NM 199129.1 | 16.67% | 50% | 909.687517 | 1262.53088 | 0.03689584 | Transmembrane protein 189 |
| NP_000205.1 | 16.67% | 72.73% | 51919.1052 | 61117.9457 | 0.00109945 | JAG1/JAGL1/CD339 Protein |
| NP 000408.1 | 16.67% | 68.18% | 56117.1426 | 64188.1274 | 0.0025987 | IL2Ra/CD25 Protein |
| NP 000582.1 | 16.67% | 72.73% | 49196.9142 | 57782.0829 | 0.00109945 | CD 14 Protein |
| NP 000868.1 | 16.67% | 72.73% | 51466.8807 | 63242.0236 | 0.00109945 | IL1R1/CD 121a Protein |
| NP_001018016.1 | 16.67% | 72.73% | 53737.3637 | 63261.689 | 0.00109945 | Mucin-1/MUC-1 Protein (Fc Tag) |
| NP_001108225.1 | 16.67% | 63.64% | 57181.0678 | 64209.546 | 0.0055972 | Endoglin/CD 105/ENG Protein |
| NPOO 1183.2 | 16.67% | 68.18% | 33528.8887 | 41882.1955 | 0.0025987 | TNFRSF17/BCMA/CD269 Protein |
| NP_001775.2 | 16.67% | 68.18% | 56511.0379 | 64611.5322 | 0.0025987 | CD97 Protein |
| NP 001954.2 | 16.67% | 68.18% | 53789.7786 | 65004.6823 | 0.0025987 | EGF/Epidermal Growth Factor Protein |
| NP 002167.1 | 16.67% | 72.73% | 48630.2868 | 57263.4877 | 0.00109945 | Interferon beta/IFN-beta/IFNB Protein |
| NP 002174.1 | 16.67% | 72.73% | 57180.8465 | 67162.1144 | 0.00109945 | IL3RA/CD 123 Protein |
| NP_003833.3 | 16.67% | 63.64% | 48845.1411 | 55552.307 | 0.0055972 | TNFRSF10B/TRAILR2/CD262 Protein |
| NP 004084.1 | 16.67% | 63.64% | 67075.0351 | 73317.2316 | 0.0055972 | EphrinB2/EFNB2 Protein |
| NP_004834.1 | 16.67% | 72.73% | 51263.7699 | 60347.4354 | 0.00109945 | IL27Ra/TCCR/WSX1 Protein |
| NP_006262.1 | 25% | 86.36% | 49594.7141 | 58104.2789 | 0.00029126 | S100Al Protein |
| NP 054862.1 | 16.67% | 68.18% | 51758.1762 | 58956.1544 | 0.0025987 | PD-L1 Protein |
| NP 061947.1 | 16.67% | 68.18% | 58248.5808 | 65031.6355 | 0.0025987 | DLL4 Protein |
| NP 068576.1 | 16.67% | 72.73% | 52394.6963 | 61677.3535 | 0.00109945 | ACE2/ACEH Protein |
| NP_079515.2 | 8.33% | 63.64% | 51984.9227 | 62947.9699 | 0.0006473 | PD-L2/B7-DC/CD273 Protein |
| P01566 | 16.67% | 72.73% | 55034.8723 | 64785.6783 | 0.00109945 | Interferon alpha 10/IFNA10 Protein |
| P01567 | 25% | 81.82% | 56287.6763 | 66801.0376 | 0.00097424 | Interferon alpha 7/IFNA7 Protein |
| PV3835 | 8.33% | 50% | 256.498926 | 748.716233 | 0.00614931 | MLCK protein (MLCK) |
| XM_376764.2 | 8.33% | 36.36% | 780.890847 | 1308.4105 | 0.0380784 | paraneoplastic antigen MA2 (PNMA2) |

TABLE 3
Histological description

| | AMVR without anti-HLA | AMVR with anti-HLA | |
|---|---|---|---|
| Histological lesions | DSA, N = 38 | DSA, N = 20 | P |
| Glomerulitis (g) | | | |
| % with g score > 0 | 38 (100.0%) | 18 (90.0%) | 0.11 |
| g score, mean ± SD | 2.1 ± 0.8 | 1.7 ± 0.9 | 0.18 |
| Peritubular capillaritis (ptc) | | | |
| % with ptc score > 0 | 36 (94.7%) | 19 (95.0) | 1.0 |
| pct score, mean ± SD | 2.0 ± 0.9 | 1.7 ± 0.7 | 0.66 |
| C4d deposition (C4d) | | | |
| % with C4d score > 0 | 9 (23.7%) | 3 (15.0%) | 0.52 |
| C4d score, mean ± SD | 0.5 ± 1.1 | 0.540.8 | 0.98 |
| Interstitial infiltrates (i) | | | |
| % with i score > 0 | 21 (55.3%) | 2 (10.0%) | 0.0008 |
| i score, mean ± SD | 0.9 ± 1.0 | 0.1 ± 0.3 | 0.003 |
| Tubulitis (t) | | | |
| % with t score > 0 | 14 (36.8%) | 14 (70.0%) | 0.03 |
| t score, mean ± SD | 1.1 ± 1.1 | 0.540.7 | 0.02 |
| TCMR diagnosis criteria, n (%) | 8 (21.1%) | 2 (10.0%) | 0.18 |
| IA, n (%) | 3 (8.8%) | 2 (10.0%) | 0.29 |
| IB, n (%) | 3 (8.8%) | 0 (0%) | 0.27 |
| IIA, n (%) | 0 (0%) | 0 (0%) | 1.00 |
| IIB, n (%) | 1 (2.6%) | 0 (0%) | 1.00 |
| III, n (%) | 1 (2.6%) | 0 (0%) | 1.00 |
| Vasculitis (v) | | | |
| % with v score > 0 | 23 (60.5%) | 3 (15.0%) | 0.001 |
| v score, mean ± SD | 1.3 ± 1.1 | 0.3 ± 0.8 | 0.0003 |
| Interstitial hemorrhages, n (%) | 12 (31.6) | 3 (15.0) | 0.22 |
| Thrombotic microangiopathy, n (%) | 6 (15.8) | 0 (0.0) | 0.08 |
| Allograft glomerulopathy (cg) | | | |
| % with cg score > 0 | 0 (0.0%) | 0 (0.0%) | 1.00 |
| eg score, mean ± SD | 0.0 ± 0.0 | 0.0 ± 0.0 | 1.00 |
| Mesangial expansion (mm) | | | |
| % with mm score > 0 | 2 (5.3%) | 0 (0.0%) | 0.54 |
| mm score, mean ± SD | 0.1 ± 0.4 | 0.0 ± 0.0 | 0.59 |
| Interstitial fibrosis (ci) | | | |
| % with ci score > 0 | 4 (10.5%) | 4 (20.0%) | 0.43 |
| ci score, mean ± SD | 0.2 ± 0.7 | 0.3 ± 0.6 | 0.97 |
| Tubular atrophy (ct) | | | |
| % with ct score > 0 | 4 (10.5%) | 4 (20.0%) | 0.42 |
| ct score, mean ± SD | 0.2 ± 0.7 | 0.2 ± 0.4 | 0.80 |
| Chronic vascular changes (cv) | | | |
| % with cv score > 0 | 16 (42.1%) | 13 (65.0%) | 0.16 |
| cv score, mean ± SD | 1.0 ± 1.1 | 0.9 ± 1.1 | 0.87 |
| Arteriolar hyalinosis (ah) | | | |
| % with ah score > 0 | 15 (39.5%) | 11 (55.5%) | 0.28 |
| ah score, mean ± SD | 0.8 ± 0.9 | 0.8 ± 1.1 | 0.59 |

TABLE 4
Top 20 immunogenic antigens in AMVR patients out of 857 candidate antigens overexpressed in microvascular ECs

| | | Delta | | | | | | | | |
|---|---|---|---|---|---|---|---|---|---|---|
| | | expression | | | | | | | | |
| | | in Micro | | | | | | | | |
| | | ECs vs | | | Frequency | Frequency | Intensity | Intensity | Overall | P |
| Gene ENS | Symbol | Macro ECs | Protein Locus | Description | in Stable | in AMVR | Stable | AMVR | score | Value |
| ENSG00000162078 | ZG16B | 148.5 | NM_145252.1 | zymogen granule | 33.33% | 90.91% | 2.11 | 2.92 | 87.2 | 0.0004 |
| | | | | protein 16B | | | | | | |
| ENSG00000163431 | LMODI | 144.8 | BC001755.1 | leiomodin 1 | 25.00% | 68.18% | 1.04 | 1.99 | 60.4 | 0.0131 |
| | | | | bone | | | | | | 0.0011 |
| ENSG00000107779 | BMPRIA | 1782.7 | NM_004329 | morphogenetic | 16.67% | 72.73% | 0.87 | 1.03 | 57.4 | |
| | | | | protein receptor, | | | | | | |
| | | | | type IA | | | | | | |
| ENSG00000197971 | MBP | 4251.9 | NM_001025100.1 | myelin basic | 25.00% | 63.64% | 1.11 | 2.16 | 56.4 | 0.0251 |
| | | | | protein | | | | | | |
| ENSG00000169188 | APEX2 | 23.7 | NM_014481.2 | APEX nuclease 2 | 25.00% | 77.27% | 0.93 | 1.09 | 55.0 | 0.0027 |
| | | | | coronin, actin | | | | | | |
| ENSG00000106789 | CORO2A | 433 | NM_052820.1 | binding protein, | 33.33% | 63.64% | 1.01 | 2.34 | 51.1 | 0.1810 |
| | | | | 2A | | | | | | |
| | | | | collagen and | | | | | | |
| ENSG00000183287 | CCBE1 | 15174.7 | BC046645.1 | calcium binding | 8.33% | 45.46% | 0.60 | 1.59 | 46.0 | 0.0621 |
| | | | | EGF domains 1 | | | | | | |
| ENSG00000145242 | EPHA5 | 1609.2 | PV3359 | EPH receptor A5 | 25.00% | 63.64% | 0.90 | 1.23 | 44.0 | 0.0771 |
| ENSG00000106829 | TLE4 | 1281.9 | BC059405.1 | transducin-like | 33.33% | 68.18% | 2.05 | 1.99 | 43.5 | 0.0451 |
| | | | | enhancer of split 4 | | | | | | |
| ENSG00000142459 | EVI5L | 1270.2 | NM_145245.1 | ecotropic viral | 25.00% | 59.09% | 0.84 | 1.33 | 41.4 | 0.1225 |
| | | | | integration site 5- | | | | | | |
| | | | | like | | | | | | |
| ENSG00000107679 | PLEKHAI | 2978.9 | NM 001001974.1 | pleckstrin | 16.67% | 45.46% | 0.69 | 1.71 | 39.7 | 0.0621 |
| | | | | homology domain | | | | | | |
| | | | | containing, family | | | | | | |
| | | | | Al | | | | | | |
| ENSG00000198959 | TGM2 | 28594.6 | BC003551.1 | transglutaminase 2 | 8.33% | 45.46% | 0.68 | 0.96 | 37.6 | 0.0621 |
| ENSG00000082805 | ERC1 | 2673.4 | PV3626 | ELKS/RAB6- | 8.33% | 31.82% | 0.39 | 2.31 | 36.0 | 0.0653 |
| | | | | interacting/CAST | | | | | | |
| | | | | family member 1 | | | | | | |
| ENSG00000198081 | ZBTB14 | 285.7 | NM_003409.2 | zinc finger and | 33.33% | 63.64% | 0.94 | 1.25 | 36.0 | 0.1810 |
| | | | | BTB domain | | | | | | |
| | | | | containing 14 | | | | | | |
| ENSG00000128872 | TMOD2 | 560.4 | BC036184.1 | tropomodulin 2 | 16.67% | 50.00% | 0.85 | 1.09 | 35.6 | 0.0369 |
| ENSG00000168175 | MAPK1IP1L | 3083.3 | NM_144578.1 | mitogen-activated | 8.33% | 40.91% | 0.60 | 1.12 | 35.5 | 0.0215 |
| | | | | protein kinase 1 | | | | | | |
| | | | | interacting protein | | | | | | |
| | | | | 1-like | | | | | | |
| ENSGOOOOO112561 | TFEB | 171.1 | NM_007162.1 | transcription | 8.33% | 40.91% | 0.62 | 0.99 | 33.7 | 0.0215 |
| | | | | factor EB | | | | | | |
| | | | | 6-phosphofructo- | | | | | | 0.1183 |
| ENSG00000123836 | PFKFB2 | 596.2 | NM_006212.1 | 2-kinase/fructose- | 41.67% | 68.18% | 1.09 | 1.40 | 33.4 | |
| | | | | 2,6-biphosphatase | | | | | | |
| | | | | 2 | | | | | | |
| ENSGOOOOO 106123 | EPHB6 | 119.4 | NM_004445.1 | EPH receptor B6 | 8.33% | 36.36% | 0.69 | 1.14 | 30.6 | 0.1540 |
| ENSG00000240694 | PNMA2 | 684.4 | XM_376764.2 | paraneoplastic Ma | 8.33% | 36.36% | 0.65 | 1.10 | 30.3 | 0.0381 |
| | | | | antigen 2 | | | | | | |

1. An in vitro method for determining the likelihood of occurrence of an acute microvascular rejection (AMVR) against a renal allograft in an individual, comprising the steps of:
a) incubating human glomerular endothelial cells with a sample of an individual under conditions wherein anti-HLA antibodies do not bind to the said human glomerular endothelial cells,
b) measuring the seroreactivity level of the said sample against the said glomerular endothelial cells,
c) comparing the seroreactivity level obtained at step b) with a reference value,
d) determining the likelihood of occurrence of an acute microvascular rejection (AMVR) against a renal allograft in the said individual based on the comparison of step c).

2. The in vitro method according to claim 1, wherein the glomerular endothelial cells of step a) do not express HLA antigens.

3. The in vitro method according to claim 1, wherein the sample used at step a) has been previously depleted in anti-HLA antibodies.

4. The in vitro method according to claim 1, wherein the individual's sample at step a) is selected in the group consisting of whole blood, blood plasma and blood serum.

5. The in vitro method according to claim 1, wherein the individual is selected from the group consisting of (i) a candidate individual for a renal allograft and (ii) a recipient of a renal allograft.

6. The in vitro method according to claim 1, wherein the said human glomerular endothelial cells consist of a human glomerular endothelial cell line.

7. The in vitro method according to claim 6, wherein the HLA antigens encoding genes of the said human glomerular endothelial cell line are inactivated.

8. An in vitro method for determining the likelihood of occurrence of an acute microvascular rejection (AMVR) against a renal allograft in an individual, comprising the steps of:
a) measuring, in a sample previously collected from the said individual, the levels of antibodies directed against one or more target antigens selected in the group consisting of ZG16B, LMOD1, BMPR1A, MBP, APEX2, CORO2A, CCBE1, EPHA5, TLE4, EV15L, PLEKHA1, TGM2, ERC1, ZBTB14, TMOD2, MAPK1IP1L, TFEB, PFKFB2, EPHB6 and PNMA2,
b) comparing each antibody level measured at step a) with a reference value,
c) determining the likelihood of occurrence of an acute microvascular rejection (AMVR) against a renal allograft in the said individual based on the comparison of step b).

9. The in vitro method according to claim 8, wherein step a) consists of measuring the levels of antibodies directed against one or more target antigens selected in the group consisting of ZG16B, LMOD1, MBP, TGM2 and PLEKHA1.

10. The in vitro method according to claim 8, wherein at step b) the reference value is the level of antibodies directed against a target antigen previously measured in renal allograft recipient individuals with no occurrence of AMVR, or against a pool serum of healthy volunteers.

11. The in vitro method according to claim 8, wherein the individual's sample at step a) is selected in the group consisting of whole blood, blood plasma and blood serum.

12. The in vitro method according to claim 8, wherein the individual is selected from the group consisting of (i) a candidate individual for a renal allograft and (ii) a recipient of a renal allograft.

13. A kit for determining the likelihood of occurrence of an acute microvascular rejection (AMVR) against a renal allograft in an individual comprising:
(i) one or more immobilized target antigens selected in the group consisting of ZG16B, LMOD1, BMPR1A, MBP, APEX2, CORO2A, CCBE1, EPHA5, TLE4, EV15L, PLEKHA1, TGM2, ERC1, ZBTB14, TMOD2, MAPK1IP1L, TFEB, PFKFB2, EPHB6 and PNMA2, and
(ii) means to detect and/or quantify the levels of antibodies directed against the immobilized target antigens in a sample previously collected from the individual.

14. Kit for determining the likelihood of occurrence of an acute microvascular rejection (AMVR) against a renal allograft in an individual comprising:
(i) immobilized human glomerular endothelial cells that under conditions wherein anti-HLA antibodies do not bind to the said human glomerular endothelial cells, and
(ii) means to detect and/or quantify the seroreactivity level of a sample previously collected from the individual against the glomerular endothelial cells.

15. Kit according to claim 14, wherein the seroreactivity level is measured against a reference value corresponding to the level of antibodies directed against a target antigen previously measured in renal allograft recipient individuals with no occurrence of AMVR or against a pool serum of healthy volunteers.

16. Use of a kit for determining the likelihood of occurrence of an acute microvascular rejection (AMVR) against a renal allograft in an individual, said kit comprising:
(i) one or more immobilized target antigens selected in the group consisting of ZG16B, LMOD1, BMPR1A, MBP, APEX2, CORO2A, CCBE1, EPHA5, TLE4, EV15L, PLEKHA1, TGM2, ERC1, ZBTB14, TMOD2, MAPK1IP1L, TFEB, PFKFB2, EPHB6 and PNMA2, and
(ii) means to detect and/or quantify the levels of antibodies directed against the immobilized target antigens in a sample previously collected from the individual.

17. Use of a kit for determining the likelihood of occurrence of an acute microvascular rejection (AMVR) against a renal allograft in an individual, said kit comprising:
(i) immobilized human glomerular endothelial cells under conditions wherein anti-HLA antibodies do not bind to the said human glomerular endothelial cells, and
(ii) means to detect and/or quantify the seroreactivity level of a sample previously collected from the individual against the glomerular endothelial cells.

18. The in vitro method according to claim 4, wherein the individual's sample at step a) is chosen from blood plasma and blood serum.

19. The in vitro method according to claim 11, wherein the individual's sample at step a) is chosen from blood plasma and blood serum.

20. A method for treating acute microvascular rejection (AMVR) in an individual who has received or who is likely to receive a renal allograft, comprising the steps of
a) determining the likelihood of occurrence of an acute microvascular rejection (AMVR) against a renal allograft in the said individual by
(1) measuring, in a sample previously collected from the said individual, the levels of antibodies directed against one or more target antigens selected in the group consisting of ZG16B, LMOD1, BMPR1A, MBP, APEX2, CORO2A, CCBE1, EPHA5, TLE4, EV15L, PLEKHA1, TGM2, ERC1, ZBTB14, TMOD2, MAPK1IP1L, TFEB, PFKFB2, EPHB6 and PNMA2;
(2) comparing each antibody level measured at step a) with a reference value;
(3) determining the likelihood of occurrence of an acute microvascular rejection (AMVR) against a renal allograft in the said individual based on the comparison of step (2)
b) selecting the said individual when the said individual has been determined as being likely to develop an acute microvascular rejection (AMVR) at step a);
c) treating the individual selected at step b) with an appropriate therapeutic treatment capable of diminishing the risk of occurrence of acute microvascular rejection (AMVR).

21. A method for treating acute microvascular rejection (AMVR) in an individual who has received or who is likely to receive a renal allograft, comprising the steps of:
a) determining the likelihood of occurrence of an acute microvascular rejection (AMVR) against a renal allograft in the said individual by
(1) incubating human glomerular endothelial cells with a sample of an individual under conditions wherein anti-HLA antibodies do not bind to the said human glomerular endothelial cells;
(2) measuring the seroreactivity level of the said sample against the said glomerular endothelial cells;
(3) comparing the seroreactivity level obtained at step (2) with a reference value, and
(4) determining the likelihood of occurrence of an acute microvascular rejection (AMVR) against a renal allograft in the said individual based on the comparison of step (3);
b) selecting the said individual when the said individual has been determined as being likely to develop an acute microvascular rejection (AMVR) at step a);
c) treating the individual selected at step b) with an appropriate therapeutic treatment capable of diminishing the risk of occurrence of acute microvascular rejection (AMVR).
